(19) United States
(12) Patent Application Publication
(10) Pub. No.: US 2005/0074851 A1
(43) Pub. Date: Apr. 7, 2005

(54) Vesicle associated proteins

(75) Inventors: Preeti Lal, Sunnyvale, CA (US); Henry Yue, Sunnyvale, CA (US); Jennifer L. Hillman, Mountain View, CA (US); Mariah R. Baughn, San Leandro, CA (US); Y. Tom Tang, San Jose, CA (US); Dyung Aina M. Lu, San Jose, CA (US); Yalda Azimzai, Castro Valley, CA (US)

Correspondence Address: FOLEY & LARDNER, P.O. BOX 80278, SAN DIEGO, CA, 92138-0278, US

(73) Assignee: INCYTE CORPORATION

(21) Appl. No.: 10/898,142

(22) Filed: Jul. 22, 2004

Related U.S. Application Data
(62) Division of application No. 09/958,460, filed on Oct. 5, 2001, now abandoned.
(60) Provisional application No. 60/144,701, filed on Jul. 20, 1999.
(60) Provisional application No. 60/128,193, filed on Apr. 7, 1999.

(51) Int. Cl. C07H 21/04; C07K 14/47

(52) U.S. Cl. 435/69.1; 435/320.1; 435/325; 530/350; 536/23.5

(57) ABSTRACT

The invention provides human vesicle associated proteins (VEAS) and polynucleotides which identify and encode VEAS. The invention also provides expression vectors, host cells, antibodies, agonists, and antagonists. The invention also provides methods for diagnosing, treating, or preventing disorders associated with expression of VEAS.

### TECHNICAL FIELD

This invention relates to nucleic acid and amino acid sequences of vesicle associated proteins and to the use of these sequences in the diagnosis, treatment, and prevention of transport disorders, autoimmune/inflammatory disorders, and cancer.

### BACKGROUND OF THE INVENTION

Eukaryotic cells are bound by a lipid bilayer membrane and subdivided into functionally distinct, membrane-bound compartments. The membranes maintain the essential differences between the cytosol, the extracellular environment, and the lumenal space of each intracellular organelle. As lipid membranes are highly impermeable to most polar molecules, transport of essential nutrients, metabolic waste products, cell signaling molecules, macromolecules, and proteins across lipid membranes and between organelles must be mediated by a variety of transport-associated molecules.

Integral membrane proteins, secreted proteins, and proteins destined for the lumen of organelles are synthesized within the endoplasmic reticulum (ER), delivered to the Golgi complex for post-translational processing and sorting, and then transported to specific intracellular and extracellular destinations. Material is internalized from the extracellular environment by endocytosis, a process essential for transmission of neuronal, metabolic, and proliferative signals; uptake of many essential nutrients; and defense against invading organisms. This intracellular and extracellular movement of protein molecules is termed vesicle trafficking. Trafficking is accomplished by the packaging of protein molecules into specialized vesicles which bud from the donor organelle membrane and fuse to the target membrane (Rothman, J. E and Wieland, F. T. (1996) Science 272:227-234).

Several steps in the transit of material along the secretory and endocytic pathways requires the formation of transport vesicles. Specifically, vesicles form at the transitional endoplasmic reticulum (tER), the rim of Golgi cisternae, the face of the Trans-Golgi Network (TGN), the plasma membrane (PM), and tubular extensions of the endosomes. Vesicle formation occurs when a region of membrane buds off from the donor organelle. The membrane-bound vesicle contains proteins to be transported and is surrounded by a proteinaceous coat, the components of which are recruited from the cytosol. Vesicle formation begins with the budding of a vesicle out of a donor organelle. The initial budding and coating processes are controlled by a cytosolic ras-like GTP-binding protein, ADP-ribosylating factor (Arf), and adapter proteins (AP). Different isoforms of both Arf and AP are involved at different sites of budding. For example, Arfs 1, 3, and 5 are required for Golgi budding, Arf4 for endosomal budding, and Arf6 for plasma membrane budding. Two different classes of coat protein have also been identified. Clathrin coats form on vesicles derived from the TGN and PM, whereas coatomer (COP) coats form on vesicles derived from the ER and Golgi. (Mellman, I. (1996) Annu. Rev. Cell Dev. Biol. 12:575-625.)

Vesicle formation begins when an adapter protein (AP) interacts with cargo proteins within the donor membrane and recruits clathrin to the bud site. APs are heterotetrameric complexes composed of two large chains (α, γ, δ, or ε, and β), a medium chain (μ), and a small chain (σ). Clathrin binds to APs via the carboxy-terminal appendage domain of the β-adaptin subunit (Le Bourgne, R. and Hoflack, B. (1998) Curr. Opin. Cell. Biol. 10:499-503). AP-1 functions in protein sorting from the TGN and endosomes to compartments of the endosomal/lysosomal system. AP-2 functions in clathrin-mediated endocytosis at the plasma membrane, while AP-3 is associated with endosomes and/or the TGN and recruits integral membrane proteins for transport to lysosomes and lysosome-related organelles. The recently isolated AP-4 complex localizes to the TGN or a neighboring compartment and may play a role in sorting events thought to take place in post-Golgi compartments (Dell' Angelica, E. C. et al. (1999) J. Biol. Chem. 274:7278-7285). Cytosolic GTP-bound Arf is also incorporated into the vesicle as it forms. Another GTP-binding protein, dynamin, forms a ring complex around the neck of the forming vesicle and provides the mechanochemical force required to release the vesicle from the donor membrane. The coated vesicle complex is then transported through the cytosol. During the transport process, Arf-bound GTP is hydrolyzed to GDP and the coat dissociates from the transport vesicle. (West, M. A. et al. (1997) J. Cell Biol. 138:1239-1254.)

Coat protein (COP) coats form on the ER and Golgi. COP coats can further be distinguished as COPI, involved in retrograde traffic through the Golgi to the ER, and COPII, involved in anterograde traffic from the ER to the Golgi. The COP coat consists of two major components, a GTP-binding protein (Arf or Sar) and coat protomer (coatomer). Coatomer is an equimolar complex of seven proteins, termed alpha-, beta-, beta′-, gamma-, delta-, epsilon- and zeta-COP. The coatomer complex binds to dilysine motifs contained on the cytoplasmic tails of integral membrane proteins. These include the dilysine-containing retrieval motif of membrane proteins of the ER and dibasic/diphenylamine motifs of members of the p24 family. The p24 family of type I membrane proteins represent the major membrane proteins of COPI vesicles. (Harter, C. and Wieland, F. T. (1998) Proc. Natl. Acad. Sci. USA 95:11649-11654.)

Vesicles can undergo homotypic or heterotypic fusion. Molecules required for appropriate targeting and fusion of vesicles include proteins in the vesicle membrane, the target membrane, and proteins recruited from the cytosol. During budding of the vesicle from the donor compartment, an integral membrane protein. VAMP (vesicle-associated membrane protein) is incorporated into the vesicle. Soon after the vesicle uncoats, a cytosolic prenylated GTP-binding protein. Rab, is inserted into the vesicle membrane. The amino acid sequence of Rab proteins reveals conserved GTP-binding domains characteristic of Ras superfamily members. In the vesicle membrane, GTP-bound Rab interacts with VAMP. Once the vesicle reaches the target membrane, a GTPase activating protein (GAP) in the target membrane converts the Rab protein to the GDP-bound form. A cytosolic protein, guanine-nucleotide dissociation inhibitor (GDI) then removes GDP-bound Rab from the vesicle membrane. Several Rab isoforms have been identified and appear to associate with specific compartments within the cell. For example, Rabs 4, 5, and 11 are associated with the early endosome, whereas Rabs 7 and 9 associate with the late endosome. These differences may provide selectivity in the association between vesicles and their target membranes. (Novick, P., and Zerial, M. (1997) Cur. Opin. Cell Biol. 9:496-504.)

Docking of the transport vesicle with the target membrane involves the formation of a complex between the vesicle SNAP receptor (v-SNARE), target membrane (t-) SNAREs, and certain other membrane and cytosolic proteins. Many of these other proteins have been identified although their exact functions in the docking complex remain uncertain. (Tellam, J. T. et al. (1995) J. Biol. Chem. 270:5857-63; Hata, Y. and Sudhof, T. C. (1995) J. Biol. Chem. 270:13022-28.) N-ethylmaleimide sensitive factor (NSF) and soluble NSF-attachment protein (α-SNAP and β-SNAP) are two such proteins that are conserved from yeast to man and function in most intracellular membrane fusion reactions. Sec1 represents a family of yeast proteins that function at many different stages in the secretory pathway including membrane fusion. Recently, mammalian homologs of Sec 1, called Munc-18 proteins, have been identified. (Katagiri, H. et al. (1995) J. Biol. Chem. 270:4963-66; Hata et al. supra.)

The SNARE complex involves three SNARE molecules, one in the vesicular membrane and two in the target membrane. Together they form a rod-shaped complex of four α-helical coiled-coils. The membrane anchoring domains of all three SNAREs project from one end of the rod. This complex is similar to the rod-like structures formed by fusion proteins characteristic of the enveloped viruses, such as myxovirus, influenza, filovirus (Ebola), and the HIV and SIV retroviruses. (Skehel, J. J., and Wiley, D. C. (1998) Cell 95:871-874.) It has been proposed that the SNARE complex is sufficient for membrane fusion, suggesting that the proteins which associate with the complex provide regulation over the fusion event. (Weber, T. et al. (1998) Cell 92:759-772.) For example, in neurons, which exhibit regulated exocytosis, docked vesicles do not fuse with the presynaptic membrane until depolarization, which leads to an influx of calcium. (Bennett, M. K., and Scheller, R. H. (1994) Annu. Rev. Biochem. 63:63-100.) Synaptotagmin, an integral membrane protein in the synaptic vesicle, associates with the t-SNARE syntaxin in the docking complex. Synaptotagmin binds calcium in a complex with negatively charged phospholipids, which allows the cytosolic SNAP protein to displace synaptotagmin from syntaxin and fusion to occur. Thus, synaptotagmin is a negative regulator of fusion in the neuron. (Littleton, J. T. et al.-(1993) Cell 74:1125-1134.) The most abundant membrane protein of synaptic vesicles appears to be the glycoprotein synaptophysin, a 38 kDa protein with four transmembrane domains. Although the function of synaptophysin is not known, its calcium-binding ability, tyrosine phosphorylation, and widespread distribution in neural tissues suggest a potential role in neurosecretiorl (Bennett, supra.)

Correct trafficking of proteins is of particular importance for the proper function of epithelial cells, which are polarized into distinct apical and basolateral domains containing different cell membrane components such as lipids and membrane-associated proteins. Certain proteins are flexible and may be sorted to the basolateral or apical side depending upon cell type or growth conditions. For example, the kidney anion exchanger (kAE 1) can be retargeted from the apical to the basolateral domain if cells are plated at higher density. The protein kanadaptin was isolated as a protein which binds to the cytoplasmic domain of kAE1. It also colocalizes with kAE1 in vesicles, but not in the membrane, suggesting that kanadaptin's function is to guide kAE1-containing vesicles to the basolateral target membrane (Chen, J. et al. (1998) J. Biol. Chem. 273:1038-1043).

The etiology of numerous human diseases and disorders can be attributed to defects in the trafficking of proteins to organelles or the cell surface. Defects in the trafficking of membrane-bound receptors and ion channels are associated with cystic fibrosis (cystic fibrosis transmembrane conductance regulator; CFTR), glucose-galactose malabsorption syndrome (Na⁺/glucose cotransporter), hypercholesterolemia (low-density lipoprotein (LDL) receptor), and forms of diabetes mellitus (insulin receptor). Abnormal hormonal secretion is linked to disorders including diabetes insipidus (vasopressin), hyper- and hypoglycemia (insulin, glucagon), Grave's disease and goiter (thyroid hormone), and Cushing's and Addison's diseases (adrenocorticotropic hormone; ACTH).

Cancer cells secrete excessive amounts of hormones or other biologically active peptides. Disorders related to excessive secretion of biologically active peptides by tumor cells include: fasting hypoglycemia due to increased insulin secretion from insulinoma-islet cell tumors; hypertension due to increased epinephrine and norepinephrine secreted from pheochromocytomas of the adrenal medulla and sympathetic paraganglia; and carcinoid syndrome, which includes abdominal cramps, diarrhea, and valvular heart disease, caused by excessive amounts of vasoactive substances (serotonin, bradykinin, histamine, prostaglandins, and polypeptide hormones) secreted from intestinal tumors. Ectopic synthesis and secretion of biologically active peptides (peptides not expected from a tumor) includes ACTH and vasopressin in lung and pancreatic cancers; parathyroid hormone in lung and bladder cancers; calcitonin in lung and breast cancers; and thyroid-stimulating hormone in medullary thyroid carcinoma.

Various human pathogens alter host cell protein trafficking pathways to their own advantage. For example, the HIV protein Nef downregulates cell-surface expression of CD4 molecules by accelerating their endocytosis through clathrin coated pits. This function of Nef is important for the spread of HIV from the infected cell (Harris, M. (1999) Curr. Biol. 9:R449-R461). A recently identified human protein, Nef-associated factor 1 (Naf1), a protein with four extended coiled-coil domains, has been found to associate with Nef. Overexpression of Naf1 increased cell surface expression of CD4, an effect which could be suppressed by Nef (Fukushi, M. et al. (1999) FEBS Lett. 442:83-88).

The discovery of new vesicle associated proteins and the polynucleotides encoding them satisfies a need in the art by providing new compositions which are useful in the diagnosis, prevention, and treatment of transport disorders, autoimmune/inflammatory disorders, and cancer.

### SUMMARY OF THE INVENTION

The invention features purified polypeptides, vesicle associated proteins, referred to collectively as “VEAS” and individually as “VEAS-1,” “VEAS-2,” “VEAS-3,” “VEAS4,” “VEAS-5,” “VEAS-6,” “VEAS-7,” “VEAS-8,” “VEAS-9,” “VEAS-10,” “VEAS-11,” “VEAS-1 2,” “VEAS-13,” “VEAS-14,” “VEAS-15,” “VEAS-16,” “VEAS-17,” “VEAS-18,” and “VEAS-19.” In one aspect, the invention provides an isolated polypeptide comprising a) an amino acid sequence selected from the group consisting of SEQ ID NO:1-19, b) a naturally occurring amino acid sequence having at least 90% sequence identity to an amino acid sequence selected from the group consisting of SEQ ID NO:1-19, c) a biologically active fragment of an amino acid sequence selected from the group consisting of SEQ ID NO:1-19, or d) an immunogenic fragment of an amino acid sequence selected from the group consisting of SEQ ID NO:1-19. In one alternative, the invention provides an isolated polypeptide comprising the amino acid sequence of SEQ ID NO:1-19.

The invention further provides an isolated polynucleotide encoding a polypeptide comprising a) an amino acid sequence selected from the group consisting of SEQ ID NO:1-19, b) a naturally occurring amino acid sequence having at least 90% sequence identity to an amino acid sequence selected from the group consisting of SEQ ID NO:1-19, c) a biologically active fragment of an amino acid sequence selected from the group consisting of SEQ ID NO:1-19, or d) an immunogenic fragment of an amino acid sequence selected from the group consisting of SEQ ID NO:1-19. In one alternative, the polynucleotide is selected from the group consisting of SEQ ID NO:20-38.

Additionally, the invention provides a recombinant polynucleotide comprising a promoter sequence operably linked to a polynucleotide encoding a polypeptide comprising a) an amino acid sequence selected from the group consisting of SEQ ID NO:1-19, b) a naturally occurring amino acid sequence having at least 90% sequence identity to an amino acid sequence selected from the group consisting of SEQ ID NO:1-19, c) a biologically active fragment of an amino acid sequence selected from the group consisting of SEQ ID NO:1-19, or d) an immunogenic fragment of an amino acid sequence selected from the group consisting of SEQ ID NO:1-19. In one alternative, the invention provides a cell transformed with the recombinant polynucleotide. In another alternative, the invention provides a transgenic organism comprising the recombinant polynucleotide.

The invention also provides a method for producing a polypeptide comprising a) an amino acid sequence selected from the group consisting of SEQ ID NO:1-19, b) a naturally occurring amino acid sequence having at least 90% sequence identity to an amino acid sequence selected from the group consisting of SEQ ID NO:1-19, c) a biologically active fragment of an amino acid sequence selected from the group consisting of SEQ ID NO:1-19, or d) an immunogenic fragment of an amino acid sequence selected from the group consisting of SEQ ID NO:1-19. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide comprising a) an amino acid sequence selected from the group consisting of SEQ ID NO:1-19, b) a naturally occurring amino acid sequence having at least 90% sequence identity to an amino acid sequence selected from the group consisting of SEQ ID NO:1-19, c) a biologically active fragment of an amino acid sequence selected from the group consisting of SEQ ID NO:1-19, or d) an immunogenic fragment of an amino acid sequence selected from the group consisting of SEQ ID NO:1-19.

The invention further provides an isolated polynucleotide comprising a) a polynucleotide sequence selected from the group consisting of SEQ ID NO:20-38, b) a naturally occurring polynucleotide sequence having at least 90% sequence identity to a polynucleotide sequence selected from the group consisting of SEQ ID NO:20-38, c) a polynucleotide sequence complementary to a), or d) a polynucleotide sequence complementary to b). In one alternative, the polynucleotide comprises at least 60 contiguous nucleotides.

Additionally, the invention provides a method for detecting a target polynucleotide in a sample, said target polynucleotide having a sequence of a polynucleotide comprising a) a polynucleotide sequence selected from the group consisting of SEQ ID NO:20-38, b) a naturally occurring polynucleotide sequence having at least 90% sequence identity to a polynucleotide sequence selected from the group consisting of SEQ ID NO:20-38, c) a polynucleotide sequence complementary to a), or d) a polynucleotide sequence complementary to b). The method comprises a) hybridizing the sample with a probe comprising at least 16 contiguous nucleotides comprising a sequence complementary to said target polynucleotide in the sample, and which probe specifically hybridizes to said target polynucleotide, under conditions whereby a hybridization complex is formed between said probe and said target polynucleotide, and b) detecting the presence or absence of said hybridization complex, and optionally, if present, the amount thereof. In one alternative, the probe comprises at least 30 contiguous nucleotides. In another alternative, the probe comprises at least 60 contiguous nucleotides.

The invention further provides a pharmaceutical composition comprising an effective amount of a polypeptide comprising a) an amino acid sequence selected from the group consisting of SEQ ID NO:1-19, b) a naturally occurring amino acid sequence having at least 90% sequence identity to an amino acid sequence selected from the group consisting of SEQ ID NO:1-19, c) a biologically active fragment of an amino acid sequence selected from the group consisting of SEQ ID NO:1-19, or d) an immunogenic fragment of an amino acid sequence selected from the group consisting of SEQ ID NO:1-19, and a pharmaceutically acceptable excipient. The invention additionally provides a method of treating a disease or condition associated with decreased expression of functional VEAS, comprising administering to a patient in need of such treatment the pharmaceutical composition.

The invention also provides a method for screening a compound for effectiveness as an agonist of a polypeptide comprising a) an amino acid sequence selected from the group consisting of SEQ ID NO:1-19, b) a naturally occurring amino acid sequence having at least 90% sequence identity to an amino acid sequence selected from the group consisting of SEQ ID NO:1-19, c) a biologically active fragment of an amino acid sequence selected from the group consisting of SEQ ID NO:1-19, or d) an immunogenic fragment of an amino acid sequence selected from the group consisting of SEQ ID NO:1-19. The method comprises a) exposing a sample comprising the polypeptide to a compound, and b) detecting agonist activity in the sample. In one alternative, the invention provides a pharmaceutical composition comprising an agonist compound identified by the method and a pharmaceutically acceptable excipient. In another alternative, the invention provides a method of treating a disease or condition associated with decreased expression of functional VEAS, comprising administering to a patient in need of such treatment the pharmaceutical composition.

Additionally, the invention provides a method for screening a compound for effectiveness as an antagonist of a polypeptide comprising a) an amino acid sequence selected from the group consisting of SEQ ID NO:1-19, b) a naturally occurring amino acid sequence having at least 90% sequence identity to an amino acid sequence selected from the group consisting of SEQ ID NO:1-19, c) a biologically active fragment of an amino acid sequence selected from the group consisting of SEQ ID NO:1-19, or d) an immunogenic fragment of an amino acid sequence selected from the group consisting of SEQ ID NO:1-19. The method comprises a) exposing a sample comprising the polypeptide to a compound, and b) detecting antagonist activity in the sample. In one alternative, the invention provides a pharmaceutical composition comprising an antagonist compound identified by the method and a pharmaceutically acceptable excipient. In another alternative, the invention provides a method of treating a disease or condition associated with overexpression of functional VEAS, comprising administering to a patient in need of such treatment the pharmaceutical composition.

The invention further provides a method for screening a compound for effectiveness in altering expression of a target polynucleotide, wherein said target polynucleotide comprises a sequence selected from the group consisting of SEQ ID NO:20-38, the method comprising a) exposing a sample comprising the target polynucleotide to a compound, and b) detecting altered expression of the target polynucleotide.

### BRIEF DESCRIPTION OF THE TABLES

Table 1 shows polypeptide and nucleotide sequence identification numbers (SEQ ID NOs), clone identification numbers (clone IDs), cDNA libraries, and cDNA fragments used to assemble full-length sequences encoding VEAS.

Table 2 shows features of each polypeptide sequence, including potential motifs, homologous sequences, and methods, algorithms, and searchable databases used for analysis of VEAS.

Table 3 shows selected fragments of each nucleic acid sequence; the tissue-specific expression patterns of each nucleic acid sequence as determined by northern analysis; diseases, disorders, or conditions associated with these tissues; and the vector into which each cDNA was cloned.

Table 4 describes the tissues used to construct the cDNA libraries from which cDNA clones encoding VEAS were isolated.

Table 5 shows the tools, programs, and algorithms used to analyze VEAS, along with applicable descriptions, references, and threshold parameters.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“VEAS” refers to the amino acid sequences of substantially purified VEAS obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of VEAS. Agonists may include proteins, nucleic acids, carbohydrates, small molecules, or any other compound or composition which modulates the activity of VEAS either by directly interacting with VEAS or by acting on components of the biological pathway in which VEAS participates.

An “allelic variant” is an alternative form of the gene encoding VEAS. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding VEAS include those sequences with deletions, insertions, or substitutions of different nucleotides, resulting in a polypeptide the same as VEAS or a polypeptide with at least one functional characteristic of VEAS. Included within this definition are polymorphisms which may or may not be readily detectable using a particular oligonucleotide probe of the polynucleotide encoding VEAS, and improper or unexpected hybridization to allelic variants, with a locus other than the normal chromosomal locus for the polynucleotide sequence encoding VEAS. The encoded protein may also be “altered,” and may contain deletions, insertions, or substitutions of amino acid residues which produce a silent change and result in a functionally equivalent VEAS. Deliberate amino acid substitutions may be made on the basis of similarity in polarity, charge, solubility, hydrophobicity, hydrophilicity, and/or the amphipathic nature of the residues, as long as the biological or immunological activity of VEAS is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of VEAS. Antagonists may include proteins such as antibodies, nucleic acids, carbohydrates, small molecules, or any other compound or composition which modulates the activity of VEAS either by directly interacting with VEAS or by acting on components of the biological pathway in which VEAS participates.

The term “antibody” refers to intact immunoglobulin molecules as well as to fragments thereof, such as Fab, F(ab′)₂, and Fv fragments, which are capable of binding an epitopic determinant. Antibodies that bind VEAS polypeptides can be prepared using intact polypeptides or using fragments containing small peptides of interest as the immunizing antigen. The polypeptide or oligopeptide used to immunize an animal (eg., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “antisense” refers to any composition capable of base-pairing with the “sense” strand of a specific nucleic acid sequence. Antisense compositions may include DNA: RNA: peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” refers to the capability of the natural, recombinant, or synthetic VEAS, or of any oligopeptide thereof, to induce a specific immune response in appropriate animals or cells and to bind with specific antibodies.

The terms “complementary” and “complementarity” refer to the natural binding of polynucleotides by base pairing. For example, the sequence “5′ A-G-T 3′” bonds to the complementary sequence “3′ T-C-A 5′.” Complementarity between two single-stranded molecules may be “partial,” such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acid strands, and in the design and use of peptide nucleic acid (PNA) molecules.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding VEAS or fragments of VEAS may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (eg., sodium dodecyl sulfate; SDS), and other components (eg., Denhardt's solution, dry milk, salmon sperm DNA, etc.).

“Consensus sequence” refers to a nucleic acid sequence which has been resequenced to resolve uncalled bases, extended using the XL-PCR kit (Perkin-Elmer, Norwalk Conn.) in the 5′ and/or the 3′ direction, and resequenced, or which has been assembled from the overlapping sequences of one or more Incyte Clones and, in some cases, one or more public domain ESTs, using a computer program for fragment assembly, such as the GELVIEW fragment assembly system (GCG, Madison Wis.). Some sequences have been both extended and assembled to produce the consensus sequence.

“Conservative amino acid substitutions” are those substitutions that, when made, least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to the chemical modification of a polypeptide sequence, or a polynucleotide sequence. Chemical modifications of a polynucleotide sequence can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “fragment” is a unique portion of VEAS or the polynucleotide encoding VEAS which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 20, 25, 30, 40, 50, 60, 75, 100, 150, 250 or at least 500 contiguous nucleotides or amino acid residues in length Fragments may be preferentially selected from certain regions of a molecule. For example, a polypeptide fragment may comprise a certain length of contiguous amino acids selected from the first 250 or 500 amino acids (or first 25% or 50% of a polypeptide) as shown in a certain defined sequence. Clearly these lengths are exemplary, and any length that is supported by the specification, including the Sequence Listing, tables, and figures, may be encompassed by the present embodiments.

A fragment of SEQ ID NO:20-38 comprises a region of unique polynucleotide sequence that specifically identifies SEQ ID NO:20-38, for example, as distinct from any other sequence in the same genome. A fragment of SEQ ID NO:20-38 is useful, for example, in hybridization and amplification technologies and in analogous methods that distinguish SEQ ID NO:20-38 from related polynucleotide sequences. The precise length of a fragment of SEQ ID NO:20-38 and the region of SEQ ID NO:20-38 to which the fragment corresponds are routinely determinable by one of ordinary skill in the art based on the intended purpose for the fragment.

A fragment of SEQ ID NO:1-19 is encoded by a fragment of SEQ ID NO:20-38. A fragment of SEQ ID NO:1-19 comprises a region of unique amino acid sequence that specifically identifies SEQ ID NO:1-19. For example, a fragment of SEQ ID NO:1-19 is useful as an immunogenic peptide for the development of antibodies that specifically recognize SEQ ID NO:1-19. The precise length of a fragment of SEQ ID NO:1-19 and the region of SEQ ID NO:1-19 to which the fragment corresponds are routinely determinable by one of ordinary skill in the art based on the intended purpose for the fragment.

The term “similarity” refers to a degree of complementarity. There may be partial similarity or complete similarity. The word “identity” may substitute for the word “similarity.” A partially complementary sequence that at least partially inhibits an identical sequence from hybridizing to a target nucleic acid is referred to as “substantially similar.” The inhibition of hybridization of the completely complementary sequence to the target sequence may be examined using a hybridization assay (Southern or northern blot, solution hybridization, and the like) under conditions of reduced stringency. A substantially similar sequence or hybridization probe will compete for and inhibit the binding of a completely similar (identical) sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% similarity or identity). In the absence of non-specific binding, the substantially similar sequence or probe will not hybridize to the second non-complementary target sequence.

The phrases “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window-4, and “diagonals saved”=4. The “weighted” residue weight table is selected as the default. Percent identity is reported by CLUSTAL V as the “percent similarity” between aligned polynucleotide sequence pairs.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.9 (May 07, 1999) set at default parameters. Such default parameters may be, for example:
Matrix: BLOSUM62
Reward for match: 1
Penalty for mismatch: −2
Open Gap: 5 and Extension Gap: 2 penalties
Gap x drop-off: 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm Methods of polypeptide sequence alignment are well-known Some alignment methods take into account conservative amino acid substitutions. Such conservative substitutions, explained in more detail above, generally preserve the hydrophobicity and acidity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.9 (May 07, 1999) with blastp set at default parameters. Such default parameters may be, for example:
Matrix: BLOSUM62
Open Gap: 11 and Extension Gap: 1 penalties
Gap x drop-off: 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size, and which contain all of the elements required for stable mitotic chromosome segregation and maintenance.

The term “humanized antibody” refers to antibody molecules in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of identity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml denatured salmon sperm DNA.

Generally, stringency of hybridization is expressed, in part, with reference to the temperature under which the wash step is carried out. Generally, such wash temperatures are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook et al., 1989, Molecular Cloning: A Laboratory Manual, 2^{nd} ed., vol. 1-3, Cold Spring Harbor Press, Plainview N.Y.: specifically see volume 2, chapter 9.

High stringency conditions for hybridization between polynucleotides of the present invention include wash conditions of 68° C. in the presence of about 0.2×SSC and about 0. 1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of VEAS which is capable of eliciting an immune response when introduced into a living organism, for example, a mammal. The term “immunogenic fragment” also includes any polypeptide or oligopeptide fragment of VEAS which is useful in any of the antibody production methods disclosed herein or known in the art.

The term “microarray” refers to an arrangement of distinct polynucleotides on a substrate.

The terms “element” and “array element” in a microarray context, refer to hybridizable polynucleotides arranged on the surface of a substrate.

The term “modulate” refers to a change in the activity of VEAS. For example, modulation may cause an increase or a decrease in protein activity, binding characteristics, or any other biological, functional, or immunological properties of VEAS.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with the second nucleic acid sequence. For instance, a promoter is operably linked to a coding sequence if the promoter affects the transcription or expression of the coding sequence. Generally, operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Probe” refers to nucleic acid sequences encoding VEAS, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30,40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook et al., 1989, Molecular Cloning: A Laboratory Manual, 2^{nd} ed., vol. 1-3, Cold Spring Harbor Press, Plainview N.Y.; Ausubel et al.,1987, Current Protocols in Molecular Biology, Greene Publ. Assoc. & Wiley-Intersciences, New York N.Y.: Innis et al., 1990, PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing nucleic acids encoding VEAS, or fragments thereof, or VEAS itself, may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acids or nucleotides by different amino acids or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

“Transformation” describes a process by which exogenous DNA enters and changes a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, viral infection, electroporation, heat shock, lipofection, and particle bombardment. The term “transformed” cells includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, and-plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 07, 1999) set at default parameters. Such a pair of nucleic acids may show, for example, at least 50%, at least 60%, at least 70%, at least 80%, at least 85%, at least 90%, at least 95% or at least 98% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 07, 1999) set at default parameters. Such a pair of polypeptides may show, for example, at least 50%, at least 60%, at least 70%, at least 80%, at least 90%, at least 95%, or at least 98% or greater sequence identity over a certain defined length of one of the polypeptides.

The Invention

The invention is based on the discovery of new human vesicle associated proteins (VEAS), the polynucleotides encoding VEAS, and the use of these compositions for the diagnosis, treatment, or prevention of transport disorders, autoimmunelinflarnmatory disorders, and cancer.

Table 1 lists the Incyte clones used to assemble full length nucleotide sequences encoding VEAS. Columns 1 and 2 show the sequence identification numbers (SEQ ID NOs) of the polypeptide and nucleotide sequences, respectively. Column 3 shows the clone IDs of the Incyte clones in which nucleic acids encoding each VEAS were identified, and column 4 shows the cDNA libraries from which these clones were isolated. Column 5 shows Incyte clones and their corresponding cDNA libraries. Clones for which cDNA libraries are not indicated were derived from pooled cDNA libraries. The Incyte clones in column 5 were used to assemble the consensus nucleotide sequence of each VEAS and are useful as fragments in hybridization technologies.

The columns of Table 2 show various properties of each of the polypeptides of the invention: column I references the SEQ ID NO; column 2 shows the number of amino acid residues in each polypeptide; column 3 shows potential phosphorylation sites; column 4 shows potential glycosylation sites; column 5 shows the amino acid residues comprising signature sequences and motifs; column 6 shows homologous sequences as identified by BLAST analysis; and column 7 shows analytical methods and in some cases, searchable databases to which the analytical methods were applied. The methods of column 7 were used to characterize each polypeptide through sequence homology and protein motifs.

The columns of Table 3 show the tissue-specificity and diseases, disorders, or conditions associated with nucleotide sequences encoding VEAS. The first column of Table 3 lists the nucleotide SEQ ID NOs. Column 2 lists fragments of the nucleotide sequences of column 1. These fragments are useful, for example, in hybridization or amplification technologies to identify SEQ ID NO:20-38 and to distinguish between SEQ ID NO:20-38 and related polynucleotide sequences. The polypeptides encoded by these fragments are useful, for example, as immunogenic peptides. Column 3 lists tissue categories which express VEAS as a fraction of total tissues expressing VEAS. Column 4 lists diseases, disorders, or conditions associated with those tissues expressing VEAS as a fraction of total tissues expressing VEAS. Column 5 lists the vectors used to subclone each cDNA library.

The columns of Table 4 show descriptions of the tissues used to construct the cDNA libraries from which cDNA clones encoding VEAS were isolated. Column 1 references the nucleotide SEQ ID NOs. column 2 shows the cDNA libraries from which these clones were isolated, and column 3 shows the tissue origins and other descriptive information relevant to the cDNA libraries in column 2.

SEQ ID NO:38 maps to chromosome 6 within the interval from 73.9 to 78.8 centiMorgans, and to chromosome 10 within the interval from 17.3 to 36.3 centiMorgans. The interval on chromosome 6 from 73.9 to 78.8 centiMorgans also contains a gene associated with hemolytic anemia due to gamma-glutamylcysteine synthetase deficiency.

The invention also encompasses VEAS variants. A preferred VEAS variant is one which has at least about 80%, or alternatively at least about 90%, or even at least about 95% arnino acid sequence identity to the VEAS amino acid sequence, and which contains at least one functional or structural characteristic of VEAS.

The invention also encompasses polynucleotides which encode VEAS. In a particular embodiment, the invention encompasses a polynucleotide sequence comprising a sequence selected from the group consisting of SEQ ID NO:20-38, which encodes VEAS. The polynucleotide sequences of SEQ ID NO:20-38, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding VEAS. In particular, such a variant polynucleotide sequence will have at least about 80%, or alternatively at least about 85%, or even at least about 95% polynucleotide sequence identity to the polynucleotide sequence encoding VEAS. A particular aspect of the invention encompasses a variant of a polynucleotide sequence comprising a sequence selected from the group consisting of SEQ ID NO:20-38 which has at least about 80%, or alternatively at least about 85%, or even at least about 95% polynucleotide sequence identity to a nucleic acid sequence selected from the group consisting of SEQ ID NO:20-38. Any one of the polynucleotide variants described above can encode an amino acid sequence which contains at least one functional or structural characteristic of VEAS.

It will be appreciated by those skilled in the art that as a result of the degeneracy of the genetic code, a multitude of polynucleotide sequences encoding VEAS, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring VEAS, and all such variations are to be considered as being specifically disclosed.

Although nucleotide sequences which encode VEAS and its variants are generally capable of hybridizing to the nucleotide sequence of the naturally occurring VEAS under appropriately selected conditions of stringency, it may be advantageous to produce nucleotide sequences encoding VEAS or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding VEAS and its derivatives without altering the encoded amino acid sequences include the production of RNA transcripts having more desirable properties, such as a greater half-life, than transcripts produced from the naturally occurring sequence.

The invention also encompasses production of DNA sequences which encode VEAS and VEAS derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding VEAS or any fragment thereof.

Also encompassed by the invention are polynucleotide sequences that are capable of hybridizing to the claimed polynucleotide sequences, and, in particular, to those shown in SEQ ID NO:20-38 and fragments thereof under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Perkin-Elmer), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Perkin-Elmer). Sequencing is then carried out using either the ABI 373 or 377 DNA sequencing system (Perkin-Elmer), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding VEAS may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA (See, eg., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker. J. D. et al. (1991) Nucleic Acids Res. 19:3055-3060). Additionally, one may use PCR, nested primers, and PROMOTERFINDER libraries (Clontech, Palo Alto Calif.) to walk genomic DNA This procedure avoids the need to screen libraries and is useful in finding intron/exon junctions. For all PCR-based methods, primers may be designed using commercially available software, such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full-length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e g., GENOTYPER and SEQUENCE NAVIGATOR, Perkin-Elmer), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode VEAS may be cloned in recombinant DNA molecules that direct expression of VEAS, or fragments or functional equivalents thereof, in appropriate host cells. Due to the inherent degeneracy of the genetic code, other DNA sequences which encode substantially the same or a functionally equivalent amino acid sequence may be produced and used to express VEAS.

The nucleotide sequences of the present invention can be engineered using methods generally known in the art in order to alter VEAS-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.: described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of VEAS, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding VEAS may be synthesized, in whole or in part, using chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, VEAS itself or a fragment thereof may be synthesized using chemical methods. For example, peptide synthesis can be performed using various solid-phase techniques. (See, e.g., Roberge, J. Y. et al. (1995) Science 269:202-204.) Automated synthesis may be achieved using the ABI 431A peptide synthesizer (Perkin-Elmer). Additionally, the amino acid sequence of VEAS, or any part thereof, may be altered during direct synthesis and/or combined with sequences from other proteins, or any part thereof, to produce a variant polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, T. (1984) Proteins, Structures and Molecular Properties, W H Freeman, New York N.Y.)

In order to express a biologically active VEAS, the nucleotide sequences encoding VEAS or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding VEAS. Such elements may vary in their strength and specificity. Specific initiation signals may also be used to achieve more efficient translation of sequences encoding VEAS. Such signals include the ATG initiation codon and adjacent sequences, e.g. the Kozak sequence. In cases where sequences encoding VEAS and its initiation codon and upstream regulatory sequences are inserted into the appropriate expression vector, no additional transcriptional or translational control signals may be needed However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing sequences encoding VEAS and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express sequences encoding VEAS. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. The invention is not limited by the host cell employed.

In bacterial systems, a number of cloning and expression vectors may be selected depending upon the use intended for polynucleotide sequences encoding VEAS. For example, routine cloning, subcloning, and propagation of polynucleotide sequences encoding VEAS can be achieved using a multifunctional E. coli vector such as PBLUESCRIPT (Stratagene, La Jolla Calif.) or PSPORT1 plasmid (Life Technologies). Ligation of sequences encoding VEAS into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of VEAS are needed, e.g. for the production of antibodies, vectors which direct high level expression of VEAS may be used For example, vectors containing the strong, inducible T5 or T7 bacteriophage promoter may be used.

Yeast expression systems may be used for production of VEAS. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of VEAS. Transcription of sequences encoding VEAS may be driven viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized In cases where an adenovirus is used as an expression vector, sequences encoding VEAS may be ligated into an adenovirus transcription/translation complex consisting of the late promoter and tripartite leader sequence. Insertion in a non-essential E1 or E3 region of the viral genome may be used to obtain infective virus which expresses VEAS in host cells. (See, e.g., Logan, J. and T. Shenk (1984) Proc. Natl. Acad Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of VEAS in cell lines is preferred. For example, sequences encoding VEAS can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-4 1 8; and als and pat confer resistance to chlorsulfuron and phosphinotricin acetyltransferase, respectively. (See, e.g., Wigler, M. et al. (1980) Proc. Natl. Acad Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding VEAS is inserted within a marker gene sequence, transformed cells containing sequences encoding VEAS can be identified by the absence of marker gene function. Alternatively, a marker gene can be placed in tandem with a sequence encoding VEAS under the control of a single promoter. Expression of the marker gene in response to induction or selection usually indicates expression of the tandem gene as well.

In general, host cells that contain the nucleic acid sequence encoding VEAS and that express VEAS may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of VEAS using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on VEAS is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding VEAS include oligolabeling, nick translation, end-labeling, or PCR amplification using a labeled nucleotide. Alternatively, the sequences encoding VEAS, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding VEAS may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode VEAS may be designed to contain signal sequences which direct secretion of VEAS through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (eg., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant nucleic acid sequences encoding VEAS may be ligated to a heterologous sequence resulting in translation of a fusion protein in any of the aforementioned host systems. For example, a chimeric VEAS protein containing a heterologous moiety that can be recognized by a commercially available antibody may facilitate the screening of peptide libraries for inhibitors of VEAS activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the VEAS encoding sequence and the heterologous protein sequence, so that VEAS may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled VEAS may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

Fragments of VEAS may be produced not only by recombinant means, but also by direct peptide synthesis using solid-phase techniques. (See, e.g., Creighton, supra, pp. 55-60.) Protein synthesis may be performed by manual techniques or by automation. Automated synthesis may be achieved, for example, using the ABI 431A peptide synthesizer (Perkin-Elmer). Various fragments of VEAS may be synthesized separately and then combined to produce the full length molecule.

Therapeutics

Chemical and structural similarity, e.g., in the context of sequences and motifs, exists between regions of VEAS and vesicle associated proteins. In addition, the expression of VEAS is closely associated with nervous tissue, cancer, inflammation/trauma and the immune response. Therefore, VEAS appears to play a role in transport disorders, autoimmune/inflammatory disorders, and cancer. In the treatment of disorders associated with increased VEAS expression or activity, it is desirable to decrease the expression or activity of VEAS. In the treatment of disorders associated with decreased VEAS expression or activity, it is desirable to increase the expression or activity of VEAS.

Therefore, in one embodiment, VEAS or a fragment or derivative thereof may be administered to a subject to treat or prevent a disorder associated with decreased expression or activity of VEAS. Examples of such disorders include, but are not limited to, a transport disorder, such as akinesia, amyotrophic lateral sclerosis, ataxia telangiectasia, cystic fibrosis, Becker's muscular dystrophy, Bell's palsy, Charcot-Marie Tooth disease, diabetes mellitus, diabetes insipidus, diabetic neuropathy, Duchenne muscular dystrophy, hyperkalemic periodic paralysis, normokalemic periodic paralysis, Parkinson's disease, malignant hyperthermia, multidrug resistance, myasthenia gravis, myotonic dystrophy, catatonia, tardive dyskinesia, dystonias, peripheral neuropathy, cerebral neoplasms, prostate cancer, cardiac disorders associated with transport, e.g., angina, bradyarrythmia, tachyarrythmia, hypertension, Long QT syndrome, myocarditis, cardiomyopathy, nemaline myopathy, centronuclear myopathy, lipid myopathy, mitochondrial myopathy, thyrotoxic myopathy, ethanol myopathy, dermatomyositis, inclusion body myositis, infectious myositis, polymyositis, neurological disorders associated with transport, e.g., Alzheimer's disease, amnesia, bipolar disorder, dementia, depression, epilepsy, Tourette's disorder, paranoid psychoses, and schizophrenia, and other disorders associated with transport, e.g., neurofibromatosis, postherpetic neuralgia, trigeminal neuropathy, sarcoidosis, sickle cell anemia, cataracts, infertility, pulmonary artery stenosis, sensorineural autosomal deafness, hyperglycemia, hypoglycemia, Grave's disease, goiter, glucose-galactose malabsorption syndrome, and hypercholesterolemia, Cushing's disease, and Addison's disease, gastrointestinal disorders including ulcerative colitis, gastric and duodenal ulcers, other conditions associated with abnormal vesicle trafficking, including acquired immunodeficiency syndrome (AIDS), allergies including hay fever, asthma, and urticaria (hives), autoimmune hemolytic anemia, proliferative glomerulonephritis, inflammatory bowel disease, multiple sclerosis, rheumatoid and osteoarthritis, scleroderma, Chediak-Higashi and Sjogren's syndromes, systemic lupus erythematosus, toxic shock syndrome, traumatic tissue damage, viral, bacterial, fungal, helminthic, and protozoal infections, cystinuria, dibasicaminoaciduria, hypercystinuria, lysinuria, hartnup disease, tryptophan malabsorption, methionine malabsorption, histidinuria, iminoglycinuria, dicarboxylicamninoaciduria, cystinosis, remal glycosuria, hypouricemia, familial hypophophatemic rickets, congenital chloridorrhea, distal renal tubular acidosis, Menkes' disease, Wilson's disease, lethal diarrhea, juvenile pernicious anemia, folate malabsorption, adrenoleukodystrophy, hereditary myoglobinuria, and Zellweger syndrome; an autoimmune/inflammatory disorder, such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; and a cancer, such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

In another embodiment, a vector capable of expressing VEAS or a fragment or derivative thereof may be administered to a subject to treat or prevent a disorder associated with decreased expression or activity of VEAS including, but not limited to, those described above.

In a further embodiment, a pharmaceutical composition comprising a substantially purified VEAS in conjunction with a suitable pharmaceutical carrier may be administered to a subject to treat or prevent a disorder associated with decreased expression or activity of VEAS including, but not limited to, those provided above.

In still another embodiment, an agonist which modulates the activity of VEAS may be administered to a subject to treat or prevent a disorder associated with decreased expression or activity of VEAS including, but not limited to, those listed above.

In a further embodiment, an antagonist of VEAS may be administered to a subject to treat or prevent a disorder associated with increased expression or activity of VEAS. Examples of such disorders include, but are not limited to, those transport disorders, autoimmune/inflammatory disorders, and cancers described above. In one aspect, an antibody which specifically binds VEAS may be used directly as an antagonist or indirectly as a targeting or delivery mechanism for bringing a pharmaceutical agent to cells or tissues which express VEAS.

In an additional embodiment, a vector expressing the complement of the polynucleotide encoding VEAS may be administered to a subject to treat or prevent a disorder associated with increased expression or activity of VEAS including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of VEAS may be produced using methods which are generally known in the art. In particular, purified VEAS may be used to produce antibodies or to screen libraries of pharmaceutical agents to identify those which specifically bind VEAS. Antibodies to VEAS may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use.

For the production of antibodies, various hosts including goats, rabbits, rats, mice, humans, and others may be immunized by injection with VEAS or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to VEAS have an amino acid sequence consisting of at least about 5 amino acids, and generally will consist of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of VEAS amino acids may be fused with those of another protein, such as KLH, and antibodies to the chimeric molecule may be produced.

Monoclonal antibodies to VEAS may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce VEAS-specific single chain antibodies. Antibodies with related specificity, but of distinct idiotypic composition, may be generated by chain shuffling from random combinatorial immunoglobulin libraries. (See, eg., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for VEAS may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between VEAS and its specific antibody. A two-site, monoclonal-based immunoassay utilizing monoclonal antibodies reactive to two non-interfering VEAS epitopes is generally used, but a competitive binding assay may also be employed (Pound, supra).

Various methods such as Scatchard analysis in conjunction with radioimmunoassay techniques may be used to assess the affinity of antibodies for VEAS. Affinity is expressed as an association constant, Kₐ, which is defined as the molar concentration of VEAS-antibody complex divided by the molar concentrations of free antigen and free antibody under equilibrium conditions. The Kₐ determined for a preparation of polyclonal antibodies, which are heterogeneous in their affinities for multiple VEAS epitopes, represents the average affinity, or avidity, of the antibodies for VEAS. The Kₐ determined for a preparation of monoclonal antibodies, which are monospecific for a particular VEAS epitope, represents a true measure of affinity. High-affinity antibody preparations with Kₐ ranging from about 10⁹ to 10¹² L/mole are preferred for use in immunoassays in which the VEAS-antibody complex must withstand rigorous manipulations. Low-affinity antibody preparations with Kₐ ranging from about 10⁶ to 10⁷ L/mole are preferred for use in immunopurification and similar procedures which ultimately require dissociation of VEAS, preferably in active form, from the antibody (Catty, D. (1988) Antibodies. Volume I: A Practical Approach, IRL Press, Washington, D.C.; Liddell, J. E. and Cryer, A. (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of VEAS-antibody complexes. Procedures for evaluating antibody specificity, titer, and avidity, and guidelines for antibody quality and usage in various applications, are generally available. (See, e.g., Catty, supra and Coligan et al. supra.)

In another embodiment of the invention, the polynucleotides encoding VEAS, or any fragment or complement thereof, may be used for therapeutic purposes. In one aspect, the complement of the polynucleotide encoding VEAS may be used in situations in which it would be desirable to block the transcription of the mRNA. In particular, cells may be transformed with sequences complementary to polynucleotides encoding VEAS. Thus, complementary molecules or fragments may be used to modulate VEAS activity, or to achieve regulation of gene function. Such technology is now well known in the art, and sense or antisense oligonucleotides or larger fragments can be designed from various locations along the coding or control regions of sequences encoding VEAS.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express nucleic acid sequences complementary to the polynucleotides encoding VEAS. (See, e.g., Sambrook, supra; Ausubel, 1995, supra.)

Genes encoding VEAS can be turned off by transforming a cell or tissue with expression vectors which express high levels of a polynucleotide, or fragment thereof, encoding VEAS. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding VEAS. Oligonucleotides derived from the transcription initiation site, e.g., between about positions −10 and +10 from the start site, may be employed. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N. Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding VEAS.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding VEAS. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a pharmaceutical or sterile composition, in conjunction with a pharmaceutically acceptable carrier, for any of the therapeutic effects discussed above. Such pharmaceutical compositions may consist of VEAS, antibodies to VEAS, and mimetics, agonists, antagonists, or inhibitors of VEAS. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, eg., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, and succinic acids. Salts tend to be more soluble in aqueous or other protonic solvents than are the corresponding free base forms. In other cases, the preparation may be a lyophilized powder which may contain any or all of the following: 1 mM to 50 mM histidine, 0.1% to 2% sucrose, and 2% to 7% mannitol, at a pH range of 4.5 to 5.5, that is combined with buffer prior to use.

After pharmaceutical compositions have been prepared, they can be placed in an appropriate container and labeled for treatment of an indicated condition. For administration of VEAS, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models such as mice, rats, rabbits, dogs, or pigs. An animal model may also be used to determine the appropriate concentration range and route of administration Such information can then be used to determine useful doses and routes for administration in humans.

A therapeutically effective dose refers to that amount of active ingredient, for example VEAS or fragments thereof, antibodies of VEAS, and agonists, antagonists or inhibitors of VEAS, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind VEAS may be used for the diagnosis of disorders characterized by expression of VEAS, or in assays to monitor patients being treated with VEAS or agonists, antagonists, or inhibitors of VEAS. Antibodies useful for diagnostic purposes may be prepared in the same manner as described above for therapeutics. Diagnostic assays for VEAS include methods which utilize the antibody and a label to detect VEAS in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring VEAS, including ELISAs, RIAS, and FACS, are known in the art and provide a basis for diagnosing altered or abnormal levels of VEAS expression Normal or standard values for VEAS expression are established by combining body fluids or cell extracts taken from normal mammalian subjects, for example, human subjects, with antibody to VEAS under conditions suitable for complex formation. The amount of standard complex formation may be quantitated by various methods, such as photometric means. Quantities of VEAS expressed in subject, control, and disease samples from biopsied tissues are compared with the standard values. Deviation between standard and subject values establishes the parameters for diagnosing disease.

In another embodiment of the invention, the polynucleotides encoding VEAS may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of VEAS may be correlated with disease. The diagnostic assay may be used to determine absence, presence, and excess expression of VEAS, and to monitor regulation of VEAS levels during therapeutic intervention.

In one aspect, hybridization with PCR probes which are capable of detecting polynucleotide sequences, including genomic sequences, encoding VEAS or closely related molecules may be used to identify nucleic acid sequences which encode VEAS. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding VEAS, allelic variants, or related sequences.

Probes may also be used for the detection of related sequences, and may have at least 50% sequence identity to any of the VEAS encoding sequences. The hybridization probes of the subject invention may be DNA or RNA and may be derived from the sequence of SEQ ID NO:20-38 or from genomic sequences including promoters, enhancers, and introns of the VEAS gene.

Means for producing specific hybridization probes for DNAs encoding VEAS include the cloning of polynucleotide sequences encoding VEAS or VEAS derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding VEAS may be used for the diagnosis of disorders associated with expression of VEAS. Examples of such disorders include, but are not limited to, a transport disorder, such as akinesia, amyotrophic lateral sclerosis, ataxia telangiectasia, cystic fibrosis, Becker's muscular dystrophy, Bell's palsy, Charcot-Marie Tooth disease, diabetes mellitus, diabetes insipidus, diabetic neuropathy, Duchenne muscular dystrophy, hyperkalemic periodic paralysis, normokalemic periodic paralysis, Parkinson's disease, malignant hyperthermia, multidrug resistance, myasthenia gravis, myotonic dystrophy, catatonia, tardive dyskinesia, dystonias, peripheral neuropathy, cerebral neoplasms, prostate cancer, cardiac disorders associated with transport, e.g., angina, bradyarrythmia, tachyarrythmia, hypertension, Long QT syndrome, myocarditis, cardiomyopathy, nemaline myopathy, centronuclear myopathy, lipid myopathy, mitochondrial myopathy, thyrotoxic myopathy, ethanol myopathy, dermatomyositis, inclusion body myositis, infectious myositis, polymyositis, neurological disorders associated with transport, e.g., Alzheimer's disease, amnesia, bipolar disorder, dementia, depression, epilepsy, Tourette's disorder, paranoid psychoses, and schizophrenia, and other disorders associated with transport, e.g., neurofibromatosis, postherpetic neuralgia, trigeminal neuropathy, sarcoidosis, sickle cell anemia, cataracts, infertility, pulmonary artery stenosis, sensorineural autosomal deafness, hyperglycemia, hypoglycemia, Grave's disease, goiter, glucose-galactose malabsorption syndrome, and hypercholesterolemnia, Cushing's disease, and Addison's disease, gastrointestinal disorders including ulcerative colitis, gastric and duodenal ulcers, other conditions associated with abnormal vesicle trafficking, including acquired immunodeficiency syndrome (AIDS), allergies including hay fever, asthma, and urticaria (hives), autoimmune hemolytic anemia, proliferative glomerulonephritis, inflammatory bowel disease, multiple sclerosis, rheumatoid and osteoarthritis, scleroderma, Chediak-Higashi and Sjogren's syndromes, systemic lupus erythematosus, toxic shock syndrome, traumatic tissue damage, viral, bacterial, fungal, helminthic, and protozoal infections, cystinuria, dibasicaminoaciduria, hypercystinuria, lysinuria, hartnup disease, tryptophan malabsorption, methionine malabsorption, histidinuria, iminoglycinuria, dicarboxylicaminoaciduria, cystinosis, renal glycosuria, hypouricemia, familial hypophophatemic rickets, congenital chloridorrhea, distal renal tubular acidosis, Menkes' disease, Wilson's disease, lethal diarrhea, juvenile pernicious anemia, folate malabsorption, adrenoleukodystrophy, hereditary myoglobinuria, and Zellweger syndrome; an autoimmune/inflammatory disorder, such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; and a cancer, such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. The polynucleotide sequences encoding VEAS may be used in Southern or northern analysis, dot blot, or other membrane-based technologies; in PCR technologies; in dipstick, pin, and multiformat ELISA-like assays; and in microarrays utilizing fluids or tissues from patients to detect altered VEAS expression. Such qualitative or quantitative methods are well known in the art.

In a particular aspect, the nucleotide sequences encoding VEAS may be useful in assays that detect the presence of associated disorders, particularly those mentioned above. The nucleotide sequences encoding VEAS may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding VEAS in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of VEAS, a normal or standard profile for expression is established. This may be accomplished by combining body fluids or cell extracts taken from normal subjects, either animal or human, with a sequence, or a fragment thereof, encoding VEAS, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding VEAS may involve the use of PCR. These oligomers may be chemically synthesized, generated enzymatically, or produced in vitro. Oligomers will preferably contain a fragment of a polynucleotide encoding VEAS, or a fragment of a polynucleotide complementary to the polynucleotide encoding VEAS, and will be employed under optimized conditions for identification of a specific gene or condition. Oligomers may also be employed under less stringent conditions for detection or quantification of closely related DNA or RNA sequences.

Methods which may also be used to quantify the expression of VEAS include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer of interest is presented in various dilutions and a spectrophotometric or colorimetric response gives rapid quantitation.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.)

In another embodiment of the invention, nucleic acid sequences encoding VEAS may be used to generate hybridization probes useful in mapping the naturally occurring genomic sequence. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.)

Fluorescent in situ hybridization (FISH) may be correlated with other physical chromosome mapping techniques and genetic map data (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding VEAS on a physical chromosomal map and a specific disorder, or a predisposition to a specific disorder, may help define the region of DNA associated with that disorder. The nucleotide sequences of the invention may be used to detect differences in gene sequences among normal, carrier, and affected individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for disease genes using positional cloning or other gene discovery techniques. Once the disease or syndrome has been crudely localized by genetic linkage to a particular genomic region, e.g., ataxia-telangiectasia to 11q22-23, any sequences mapping to that area may represent associated or regulatory genes for further investigation. (See, eg., Gatti, R. A. et al. (1988) Nature 336:577-580.) The nucleotide sequence of the subject invention may also be used to detect differences in the chromosomal location due to translocation, inversion, etc., among normal, carrier, or affected individuals.

In another embodiment of the invention, VEAS, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between VEAS and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with VEAS, or fragments thereof, and washed. Bound VEAS is then detected by methods well known in the art. Purified VEAS can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding VEAS specifically compete with a test compound for binding VEAS. In this manner, antibodies can be used to detect the presence of any peptide which shares one or more antigenic determinants with VEAS.

In additional embodiments, the nucleotide sequences which encode VEAS may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications and publications, mentioned above and below, in particular U.S. Ser. No. 60/128,193 and U.S. Ser. No. 60/144,701, are hereby expressly incorporated by reference.

### EXAMPLES

I. Construction of cDNA Libraries

RNA was purchased from Clontech or isolated from tissues described in Table 4. Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A+) RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), pcDNA2.1 plasmid (Invitrogen, Carlsbad Calif.), or pINCY plasmid (Incyte Pharmaceuticals, Palo Alto Calif.). Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids were recovered from host cells by in vivo excision using the UNIZAP vector system (Stratagene) or by cell lysis. Plasmids were purified using at least one of the following; a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

cDNA sequencing reactions were processed using standard methods or high-throughput instrumentation such as the ABI CATALYST 800 (Perkin-Elmer) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Perkin-Elmer). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Perkin-Elmer) in conjunction with standard ABI protocols and base calling software; or other sequence analysis systems known in the art. Reading frames within the cDNA sequences were identified using standard methods (reviewed in Ausubel, 1997, supra unit 7.7). Some of the cDNA sequences were selected for extension using the techniques disclosed in Example VI.

The polynucleotide sequences derived from cDNA sequencing were assembled and analyzed using a combination of software programs which utilize algorithms well known to those skilled in the art. Table 5 summarizes the tools, programs, and algorithms used and provides applicable descriptions, references, and threshold parameters. The first column of Table 5 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score, the greater the homology between two sequences). Sequences were analyzed using MACDNASIS PRO software (Hitachi Software Engineering, South San Francisco Calif.) and LASERGENE software (DNASTAR). Polynucleotide and polypeptide sequence alignments were generated using the default parameters specified by the clustal algorithm as incorporated into the MEGALIGN multisequence alignment program (DNASTAR), which also calculates the percent identity between aligned sequences.

The polynucleotide sequences were validated by removing vector, linker, and polyA sequences and by masking ambiguous bases, using algorithms and programs based on BLAST, dynamic programing, and dinucleotide nearest neighbor analysis. The sequences were then queried against a selection of public databases such as the GenBank primate, rodent, mammalian, vertebrate, and eukaryote databases, and BLOCKS, PRINTS, DOMO, PRODOM, and PFAM to acquire annotation using programs based on BLAST, FASTA, and BLIMPS. The sequences were assembled into full length polynucleotide sequences using programs based on Phred, Phrap, and Consed, and were screened for open reading frames using programs based on GeneMark, BLAST, and FASTA. The full length polynucleotide sequences were translated to derive the corresponding full length amino acid sequences, and these full length sequences were subsequently analyzed by querying against databases such as the GenBank databases (described above), SwissProt, BLOCKS, PRINTS, DOMO, PRODOM, Prosite, and Hidden Markov Model (HMM)-based protein family databases such as PFAM. HMM is a probabilistic approach which analyzes consensus primary structures of gene families. (See, e.g., Eddy, S. R. (1996) Curr. Opin. Struct. Biol. 6:361-365.)

The programs described above for the assembly and analysis of full length polynucleotide and amino acid sequences were also used to identify polynucleotide sequence fragments from SEQ ID NO:20-38. Fragments from about 20 to about 4000 nucleotides which are useful in hybridization and amplification technologies were described in The Invention section above.

IV. Northern Analysis

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7: Ausubel, 1995, supra, ch. 4 and 16.)

Analogous computer techniques applying BLAST were used to search for identical or related molecules in nucleotide databases such as GenBank or LIFESEQ (Incyte Pharmaceuticals). This analysis is much faster than multiple membrane-based hybridizations. In addition, the sensitivity of the computer search can be modified to determine whether any particular match is categorized as exact or similar. The basis of the search is the product score, which is defined as:
% sequence identity×% maximum BLAST score/100
The product score takes into account both the degree of similarity between two sequences and the length of the sequence match. For example, with a product score of 40, the match will be exact within a 1% to 2% error, and, with a product score of 70, the match will be exact. Similar molecules are usually identified by selecting those which show product scores between 15 and 40, although lower scores may identify related molecules.

The results of northern analyses are reported as a percentage distribution of libraries in which the transcript encoding VEAS occurred. Analysis involved the categorization of cDNA libraries by organ/tissue and disease. The organ/tissue categories included cardiovascular, dermatologic, developmental, endocrine, gastrointestinal, hematopoietic/immune, musculoskeletal, nervous, reproductive, and urologic. The disease/condition categories included cancer, inflammation, trauma, cell proliferation, neurological, and pooled. For each category, the number of libraries expressing the sequence of interest was counted and divided by the total number of libraries across all categories. Percentage values of tissue-specific and disease- or condition-specific expression are reported in Table 3.

V. Chromosomal Mapping of VEAS Encoding Polynucleotides

The cDNA sequences which were used to assemble SEQ ID NO:35-38 were compared with sequences from the Incyte LIFESEQ database and public domain databases using BLAST and other implementations of the Smith-Waterman algorithm. Sequences from these databases that matched SEQ ID NO:35-38 were assembled into clusters of contiguous and overlapping sequences using assembly algorithms such as Phrap (Table 5). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

The genetic map locations of SEQ ID NO:38 are described in The Invention as ranges, or intervals, of human chromosomes. More than one map location is reported for SEQ ID NO:38, indicating that previously mapped sequences having similarity, but not complete identity, to SEQ ID NO:38 were assembled into their respective clusters. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap '99” World Wide Web site (http://www.ncbi.nlm.nih.gov/genemap/), can be employed to determine if previously identified disease genes map within or in proximity to the intervals indicated above.

VI. Extension of VEAS Encoding Polynucleotides

The full length nucleic acid sequences of SEQ ID NO:20-38 were produced by extension of an appropriate fragment of the full length molecule using oligonucleotide primers designed from this fragment. One primer was synthesized to initiate 5′ extension of the known fragment, and the other primer, to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1× TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, individual colonies were picked and cultured overnight at 37° C. in 384-well plates in LB/2× carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5; steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Perkin-Elmer).

In like manner, the nucleotide sequences of SEQ ID NO:20-38 are used to obtain 5′ regulatory sequences using the procedure above, oligonucleotides designed for such extension, and an appropriate genomic library.

VII. Labeling and Use of Individual Hybridization Probes

Hybridization probes derived from SEQ ID NO:20-38 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

VIII. Microarrays

A chemical coupling procedure and an ink jet device can be used to synthesize array elements on the surface of a substrate. (See, eg., Baldeschweiler, supra.) An array analogous to a dot or slot blot may also be used to arrange and link elements to the surface of a substrate using thermal, UV, chemical, or mechanical bonding procedures. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each probe which hybridizes to an element on the microarray may be assessed through analysis of the scanned images.

Full-length cDNAs, Expressed Sequence Tags (ESTs), or fragments thereof may comprise the elements of the microarray. Fragments suitable for hybridization can be selected using software well known in the art such as LASERGENE software (DNASTAR). Full-length cDNAs, ESTs, or fragments thereof corresponding to one of the nucleotide sequences of the present invention, or selected at random from a cDNA library relevant to the present invention, are arranged on an appropriate substrate, e.g., a glass slide. The cDNA is fixed to the slide using, e.g., UV cross-linking followed by thermal and chemical treatments and subsequent drying. (See, e.g., Schena, M. et al. (1995) Science 270:467470; Shalon, D. et al. (1996) Genome Res. 6:639-645.) Fluorescent probes are prepared and used for hybridization to the elements on the substrate. The substrate is analyzed by procedures described above.

IX. Complementary Polynucleotides

Sequences complementary to the VEAS-encoding sequences, or any parts thereof, are used to detect, decrease, or inhibit expression of naturally occurring VEAS. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of VEAS. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the VEAS-encoding transcript.

X. Expression of VEAS

Expression and purification of VEAS is achieved using bacterial or virus-based expression systems. For expression of VEAS in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express VEAS upon induction with isopropyl beta-D-thiogalactopyranoside (IPTG). Expression of VEAS in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding VEAS by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, VEAS is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from VEAS at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified VEAS obtained by these methods can be used directly in the following activity assay.

XI. Demonstration of VEAS Activity

VEAS activity is measured by its inclusion in coated vesicles. VEAS can be expressed by transforming a mammalina cell line such as COS7, HeLa, or CHO with an eukaryotic expression vector encoding VEAS. Eukaryotic expression vectors are commercially available, and the techniques to introduce them into cells are well known to those skilled in the art. A small amount of a second plasmid, which expresses any one of a number of marker genes, such as β-galactosidase, is co-transformed into the cells in order to allow rapid identification of those cells which have taken up and expressed the foreign DNA. The cells are incubated for 48-72 hours after transformation under conditions appropriate for the cell line to allow expression and accumulation of VEAS and β-galactosidase.

Transformed cells are collected and cell lysates are assayed for vesicle formation A non-hydrolyzable form of GTP, GTPγS, and an ATP regenerating system are added to the lysate and the mixture is incubated at 37° C. for 10 minutes. Under these conditions, over 90% of the vesicles remain coated (Orci, L. et al. (1989) Cell 56:357-368). Transport vesicles are salt-released from the Golgi membranes, loaded under a sucrose gradient, centrifuged, and fractions are collected and analyzed by SDS-PAGE. Co-localization of VEAS with clathrin or COP coatamer is indicative of VEAS activity in vesicle formation. The contribution of VEAS in vesicle formation can be confirmed by incubating lysates with antibodies specific for VEAS prior to GTPγS addition The antibody will bind to VEAS and interfere with its activity, thus preventing vesicle formation

In the alternative, VEAS activity is measured by its ability to alter vesicle trafficking pathways. Vesicle trafficking in cells transformed with VEAS is examined using fluorescence microscopy. Antibodies specific for vesicle coat proteins or typical vesicle trafficking substrates such as transferrin or the mannose-6-phosphate receptor are commercially available. Various cellular components such as ER, Golgi bodies, peroxisomes, endosomes, lysosomes, and the plasmalemma are examined. Alterations in the numbers and locations of vesicles in cells transformed with VEAS as compared to control cells are characteristic of VEAS activity.

XII. Functional Assays

VEAS function is assessed by expressing the sequences encoding VEAS at physiologically elevated levels in mammalian cell culture systems. cDNA is subcloned into a mammalian expression vector containing a strong promoter that drives high levels of cDNA expression. Vectors of choice include pCMV SPORT plasmid (Life Technologies) and pCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake: alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of VEAS on gene expression can be assessed using highly purified populations of cells transfected with sequences encoding VEAS and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding VEAS and other genes of interest can be analyzed by northern analysis or microarray techniques.

XIII. Production of VEAS Specific Antibodies

VEAS substantially purified using polyacrylamide gel electrophoresis (PAGE; see, e g., Harrington, M. G. (1990) Methods Enzymol. 182:488-495), or other purification techniques, is used to immunize rabbits and to produce antibodies using standard protocols.

Alternatively, the VEAS amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI 431A peptide synthesizer (Perkin-Elmer) using fmoc-chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-VEAS activity by, for example, binding the peptide or VEAS to a substrate, blocking with 1% BSA, reacting with rabbit antisera, washing, and reacting with radio-iodinated goat anti-rabbit IgG.

XIV. Purification of Naturally Occurring VEAS Using Specific Antibodies

Naturally occurring or recombinant VEAS is substantially purified by immunoaffinity chromatography using antibodies specific for VEAS. An immunoaffinity column is constructed by covalently coupling anti-VEAS antibody to an activated chromatographic resin, such as CNBr-activated SEPHAROSE (Amersham Pharmacia Biotech). After the coupling, the resin is blocked and washed according to the manufacturer's instructions.

Media containing VEAS are passed over the immunoaffinity column, and the column is washed under conditions that allow the preferential absorbance of VEAS (e.g., high ionic strength buffers in the presence of detergent). The column is eluted under conditions that disrupt antibody/VEAS binding (e.g., a buffer of pH 2 to pH 3, or a high concentration of a chaotrope, such as urea or thiocyanate ion), and VEAS is collected.

XV. Identification of Molecules Which Interact with VEAS

VEAS, or biologically active fragments thereof, are labeled with ¹²⁵I Bolton-Hunter reagent. (See, e.g., Bolton A. E. and W. M. Hunter (1973) Biochem. J. 133:529-539.) Candidate molecules previously arrayed in the wells of a multi-well plate are incubated with the labeled VEAS, washed, and any wells with labeled VEAS complex are assayed. Data obtained using different concentrations of VEAS are used to calculate values for the number, affinity, and association of VEAS with the candidate molecules.

Alternatively, molecules interacting with VEAS are analyzed using the yeast two-hybrid system as described in Fields, S. and O. Song (1989, Nature 340:245-246), or using commercially available kits based on the two-hybrid system, such as the MATCHMAKER system (Clontech).

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| Protein | Nucleotide | | | |
|---|---|---|---|---|
| SEQ ID NO: | SEQ ID NO: | Clone ID | Library | Fragments |
| 1 | 20 | 665637 | SCORNOT01 | 081198F1 (SYNORAB01); 665637H1 (SCORNOT01); 758691T6 |
| | | | | (BRAITUT02); 4692726H1 (BRAENOT02) |
| 2 | 21 | 745823 | BRAITUT01 | 115845F1 (KIDNNOT01); 745823H1 (BRAITUT01); 2584841F6 |
| | | | | (BRAITUT22) |
| 3 | 22 | 776854 | COLNNOT05 | 776854H1, 776854R6 and 776854T6 (COLNNOT05); 3342008H1 |
| | | | | (SPLNNOT09); 3420545X309D1 (UCMCNOT04); 5077144F6 |
| | | | | (COLCTUT03) |
| 4 | 23 | 1273556 | TESTTUT02 | 078149F1 (SYNORAB01); 374719R6 (LUNGNOT02); 1237029F1 |
| | | | | (LUNGFET03); 1273556H1 (TESTTUT02); 1803328F6 (SINTNOT13); |
| | | | | 2685872H1 (LUNGNOT23); 3843874H1 (DENDNOT01) |
| 5 | 24 | 1505808 | BRAITUT07 | 659082R6 (BRAINOT03); 1505808H1 (BRAITUT07); 4775166H1 |
| | | | | (BRAQNOT01) |
| 6 | 25 | 1814911 | PROSNOT20 | 1702322F6 (BLADTUT05); 1747807F6 (STOMTUT02); 1814911H1 |
| | | | | (PROSNOT20); 1876186F6 (LEUKNOT02); 1979383R6 (LUNGTUT03); |
| | | | | 3497874H1 (PROSTUT13) |
| 7 | 26 | 2087812 | PANCNOT04 | 2087812H1 (PANCNOT04); 2773884F6 (PANCNOT15); 3535373T6 |
| | | | | (KIDNNOT25) |
| 8 | 27 | 2149274 | BRAINOT09 | 1522793H1 (BLADTUT04); 1631562F6 (COLNNOT19); 1755633F6 |
| | | | | (LIVRTUT01); 2098374R6 (BRAITUT02); 2149274H1 (BRAINOT09); |
| | | | | 2422887R6 (SCORNON02); 2452261F6 (ENDANOT01); 2511137X11F1 |
| | | | | (CONUTUT01); 2517384H1 (LIVRTUT04); 3278280H1 (STOMFET02); |
| | | | | 3458453H1 (293TF1T01); 3596844H1 (FIBPNOT01); 3745870H1 |
| | | | | (THYMNOT08); 3747945H1 (UTRSNOT18); 3805602H1 (BLADTUT03) |
| 9 | 28 | 2355124 | LUNGNOT20 | 555023F1 (SCORNOT01); 1753596F6 (LIVRTUT01); 2355124F6 and |
| | | | | 2355124H1 (LUNGNOT20); 2697365F6 (UTRSNOT12); 4190620H1 |
| | | | | (BRAPDIT01); 4328830F6 (KIDNNOT32) |
| 10 | 29 | 2366939 | ADRENOT07 | 483554H1 (HNT2RAT01); 665136R6 (SCORNOT01); 2366939F6, |
| | | | | 2366939H1 and 2367767X11F1 (ADRENOT07); 2457182F6 and |
| | | | | 2457182T6 (ENDANOT01); 3534278H1 (KIDNNOT25); 3640393H1 |
| | | | | (LUNGNOT30); 4997211H1 (MYEPTXT02); SAFC01017F1 |
| 11 | 30 | 2483906 | SMCANOT01 | 2483906F6 and 2483906H1 (SMCANOT01); 3589469H1 (293TF5T01) |
| 12 | 31 | 2499488 | ADRETUT05 | 077509R1 (SYNORAB01); 099726R6 (ADRENOT01); 975524H1 |
| | | | | (MUSCNOT02); 1332009F1 (PANCNOT07); 1695290F6 (COLNNOT23); |
| | | | | 1708568H1 (PROSNOT16); 2499488F6 and 2499488H1 |
| | | | | (ADRETUT05); 3038012H1 (BRSTNOT16); 3591434F6 (293TF5T01); |
| | | | | 4712957H1 (BRAIHCT01); 5372332H1 (BRAINOT22) |
| 13 | 32 | 2559148 | ADRETUT01 | 678875R6 (UTRSNOT02); 1395446F6 (THYRNOT03); 1669748F6 |
| | | | | (BMARNOT03); 1821559H1 (GBLATUT01); 2197133F6 (SPLNFET02); |
| | | | | 2559148H1 (ADRETUT01); 3132150H1 (BLADNOT08); 3334672H1 |
| | | | | (BRAIFET01); 4376567H1 (CONFNOT03) |
| 14 | 33 | 3321481 | PTHYNOT03 | 1556277X11C1 (BLADTUT04); 1998047R6 (BRSTTUT03); 3321481H1 |
| | | | | (PTHYNOT03); SAFC02175F1; SAFC02744F1 |
| 15 | 34 | 3367918 | CONNTUT04 | 3367918H1 (CONNTUT04); 284546X1 (CARDNOT01); 882035X33 |
| | | | | (THYRNOT02); 4157516H1 (ADRENOT14) |
| 16 | 35 | 1227327 | COLNNOT01 | 1223664R1 (COLNTUT02), 1223664T1 (COLNTUT02), 1227327H1 |
| | | | | (COLNNOT01), 1227327R6 (COLNNOT01), 1711230F6 (PROSNOT16), |
| | | | | 1951035H1 (PITUNOT01), 1997538R6 (BRSTTUT03), 3231314H1 |
| | | | | (COTRNOT01), 3282015H1 (STOMFET02), 4145153H1 (SINITUT04), |
| | | | | 4347830F6 (TLYMTXT01), 4414694H1 (MONOTXT01), 4691853H1 |
| | | | | (BRAENOT02) |
| 17 | 36 | 1416292 | BRAINOT12 | 160083H1 (ADENINB01), 495694T6 (HNT2NOT01), 917398H1 |
| | | | | (BRSTNOT04), 1416292H1 (BRAINOT12), 1485986F6 (CORPNOT02), |
| | | | | 1698268F6 (BLADTUT05), 1812119T6 (PROSTUT12), 1950135R6 |
| | | | | (PITUNOT01), 2968531H1 (HEAONOT02), 3769568H1 (BRSTNOT24), |
| | | | | 4555506F6 (KERAUNT01) |
| 18 | 37 | 2611704 | LUNGNOT23 | 688077T6 (UTRSNOT02), 2611704H1 (LUNGNOT23), 4414279F6 |
| | | | | (MONOTXT01) |
| 19 | 38 | 5136672 | OVARDIT04 | 067284R1 (HUVESTB01), 1350538F1 (LATRTUT02), 3393953H1 |
| | | | | (LUNGNOT28), 3684532H1 (HEAANOT01), 4970666H1 (KIDEUNC10), |
| | | | | 5136672H1 (OVARDIT04), SBIA10538D1, SBIA05310D1, |
| | | | | SBIA00959D1 |

TABLE 2

| | Amino | Potential | Potential | | | |
|---|---|---|---|---|---|---|
| Protein | Acid | Phosphorylation | glycosylation | | Homologous | Analytical |
| Seq ID NO: | Residues | Sites | sites | Signature Sequence | Sequences | Methods |
| 1 | 144 | T29 S48 T74 S99 | N72 | clathrin adapter complex- | Adaptor related | HMMER-PFAM |
| | | T126 | | small subunit: | protein complex | BLIMPS-BLOCKS |
| | | | | M1-S142; F5-K18; F66-E116 | AP-4, sigma 4 | BLAST-GenBank |
| | | | | | subunit [Mus | |
| | | | | | musculus] | |
| | | | | | g4426605 | |
| 2 | 177 | S109 S128 | N60 | ADP-ribosylation factor | ADP-ribosylation | HMMER-PFAM |
| | | | | family: | factor ARF-2 | BLIMPS-PRINTS |
| | | | | G2-G160; R19-T42; P47-K86; | [Mus musculus] | BLIMPS-BLOCKS |
| | | | | M1-G29; I33-D67 | g1565209 | BLAST-GenBank |
| 3 | 408 | S29 T54 T176 | N127 N174 | signal peptide: M1-S29 | mollusk-derived | SPSCAN |
| | | S205 S231 T268 | N185 | aromatic amino acid | growth factor | HMMER |
| | | T279 S333 S7 T65 | | permease: | [Aplysia | BLIMPS-PRINTS |
| | | T129 T139 T152 | | A18-L37; L66-H85 | californica] | BLAST-GenBank |
| | | S156 S265 S304 | | | g4235110 | |
| 4 | 553 | S130 S198 S286 | N71 N214 N308 | cell attachment seq: | lysosomal/endosomal | MOTIFS |
| | | T382 S465 S73 | N366 N411 | R466-D468 | membrane | SPSCAN |
| | | T146 S216 S272 | N526 | signal peptide: M1-A32 | protein [T. brucei] | BLAST-GenBank |
| | | S335 S449 T543 | | transmembrane domain: | g3378150 | HMMER |
| | | Y428 | | L275-T302 | | |
| 5 | 179 | T13 T22 S160 | | MAS20 protein import | SNAP regulatory | BLIMPS-PRINTS |
| | | | | receptor: | protein | BLIMPS-BLOCKS |
| | | | | E81-R93 | complexin I | BLAST-GenBank |
| | | | | | [Homo sapiens] | |
| | | | | | g2465459 | |
| 6 | 336 | T278 S87 T218 | | signal peptide: M1-S43 | peroxisomal | SPSCAN |
| | | T267 S171 S242 | | | peripheral | BLAST-GenBank |
| | | S261 | | | membrane protein | |
| | | | | | Pex16p [Yarrowia | |
| | | | | | lipolytica] | |
| | | | | | g1813611 | |
| 7 | 240 | S109 T231 S18 | N107 N156 | emp24/gp25L/p24 family | Putative T1/ST2 | HMMER-PFAM |
| | | S21 T166 T235 | | protein: | receptor binding | BLIMPS-BLOCKS |
| | | | | F6-T233; M176-F227 | protein [Mus | BLAST-GenBank |
| | | | | | musculus] | |
| | | | | | g1223892 | |
| 8 | 955 | S5 S104 T368 | N760 | Ig and MHC protein | alpha-adaptin | MOTIFS |
| | | T379 T470 T482 | | signature: F127-H133 | coated vesicle | SPSCAN |
| | | T597 S626 S636 | | signal peptide: M1-S25 | protein g55729 | BLIMPS-BLOCKS |
| | | T698 T938 S54 | | (NSF) attachment protein: | [Rattus | BLAST-GenBank |
| | | S85 T89 S163 | | G46-I65 | norvegicus] | |
| | | T189 S258 S297 | | | | |
| | | T379 S384 T470 | | | | |
| | | T787 S819 T832 | | | | |
| | | T935 T938 Y394 | | | | |
| | | Y418 Y442 | | | | |
| 9 | 247 | T35 S65 S213 T81 | N200 | signal peptide: M1-S24 | vesicular | SPScan |
| | | Y37 Y130 Y146 | | | transport | HMMER |
| | | | | | protein NIPSNAP2 | BLAST-GenBank |
| | | | | | [Homo sapiens] | |
| | | | | | g2769254 | |
| 10 | 532 | T112 S26 S200 | | PKC calcium-binding (C2) | secretory | MOTIFS |
| | | S279 S373 S432 | | domain: | vesicle-assoc. | HMMER-PFAM |
| | | T478 T527 T25 | | G147-F162; C8-S99; V140-Q230; | protein | BLIMPS-PRINTS |
| | | S60 S86 S107 | | N53-D66 | copine III [Homo | |
| | | S108 S271 S338 | | | sapiens] | |
| | | | | | g5670328 | |
| 11 | 154 | T32 T22 S48 T144 | | clathrin adapter complex- | AP-1 clathrin | MOTIFS |
| | | T126 S127 | | small chain: | adaptor complex | HMMER-PFAM |
| | | | | L57-F67; M1-Q142; E100-S127; | sigma1B subunit | BLIMPS-PRINTS |
| | | | | I5-K18; Y66-E116 | [Homo sapiens] | BLIMPS-BLOCKS |
| | | | | | g3641680 | BLAST-GenBank |
| 12 | 684 | S490 T70 S72 T94 | N225 N351 | | cytoskeleton | BLAST-GenBank |
| | | S137 T159 S211 | N663 | | associated | |
| | | S219 S266 S325 | | | intracellular | |
| | | T360 S437 S443 | | | transport | |
| | | S498 S499 S536 | | | protein | |
| | | S544 T562 S612 | | | Uso1 [S. cerevisiae] | |
| | | S613 S624 T674 | | | g4778 | |
| | | S6 S11 T133 T192 | | | | |
| | | T251 S396 S420 | | | | |
| | | T468 T533 S552 | | | | |
| | | T564 T667 | | | | |
| 13 | 576 | T249 S18 T28 T79 | N16 N389 | cell attachment sequence: | clathrin coat | MOTIFS |
| | | T87 T105 S180 | | R232-D234 | associated | BLIMPS-BLOCKS |
| | | S181 S195 S220 | | bindin precursor | protein | BLIMPS-PRINTS |
| | | S245 S256 S364 | | signature: G554-N570 | Epsin [Rattus | BLAST-GenBank |
| | | T399 S405 S420 | | | norvegicus] | |
| | | S447 S5 S89 S245 | | | g3249559 | |
| | | T467 | | | | |
| 14 | 425 | S307 S28 S102 | N76 N186 N230 | clathrin adapter complex- | AP-mu chain | MOTIFS |
| | | S247 S283 S291 | | medium chain: | family member | HMMER-PFAM |
| | | S320 T71 S102 | | V157-E177; V6-T424; G13-F33; | Mu1b, clathrin- | BLIMPS-PRINTS |
| | | S139 T144 S158 | | E100-D127; W159-G187; | associated | BLIMPS-BLOCKS |
| | | T197 S268 S324 | | L235-G262; K304-P319; | protein [Mus | BLAST-GenBank |
| | | S333 S346 Y405 | | I344-E355; Y94-L131; V157-N186; | musculus] | |
| | | | | D237-R270; Y405-R423 | g4704421 | |
| 15 | 167 | T42 T84 S139 T84 | | stomatin signature: | caveolae-assoc. | BLIMPS-BLOCKS |
| | | Y94 | | A110-E131 | membrane protein | BLIMPS-PRINTS |
| | | | | | flotillin-1 | BLAST-GenBank |
| | | | | | g3599573 [Homo | |
| | | | | | sapiens] | |
| 16 | 739 | S154 S182 S208 | | Beta adaptin signature: | AP-4 adaptor | BLAST-GenBank |
| | | S229 S321 T377 | | E12-Q625 | complex, beta4 | MOTIFS |
| | | T404 S684 S725 | | | subunit | BLAST-DOMO |
| | | T733 S111 S535 | | | [Mus musculus] | BLAST-PRODOM |
| | | S637 S709 | | | g5442364 | |
| 17 | 742 | T550 T698 S6 S41 | N191 N653 | Leucine zipper: L452-L473 | kanadaptin | BLAST-GenBank |
| | | S76 S141 S164 | | | [Mus musculus] | MOTIFS |
| | | S258 S275 S412 | | | g2661090 | |
| | | T416 S493 T540 | | | | |
| | | S658 S682 S689 | | | | |
| | | T117 S128 S214 | | | | |
| | | S267 T355 S366 | | | | |
| | | S396 S463 S499 | | | | |
| | | S709 Y333 Y410 | | | | |
| | | Y719 | | | | |
| 18 | 325 | S16 S63 T77 S87 | N232 | Coiled coil domain: | Naf1 beta | BLAST-GenBank |
| | | S175 S184 T195 | | M33-M263 | protein | MOTIFS |
| | | S216 T8 S27 T28 | | | [Homo sapiens] | BLAST-PRODOM |
| | | S63 S319 | | | g3758821 | |
| 19 | 299 | T101 S285 S60 | N271 | GNS1/SUR4 family domains: | VBM2 (v-SNARE | BLAST-GenBank |
| | | S191 S260 | | Q57-W88, Y238-Y254 | bypass mutant) | MOTIFS |
| | | | | | [S. cerevisiae] | BLIMPS-BLOCKS |
| | | | | | g2654761 | |

TABLE 3

| | Useful | | | |
|---|---|---|---|---|
| Nucleotide | Nucleotide | Tissue Expression | Disease or Condition | |
| Seq ID NO: | Fragment | (Fraction of Total) | (Fraction of Total) | Vector |
| 20 | 242-301 | Nervous (0.316) | Cancer (0.474) | PSPORT1 |
| | | Developmental (0.158) | Inflammation (0.369) | |
| | | Gastrointestinal (0.158) | Cell Proliferation (0.211) | |
| | | Reproductive (0.158) | | |
| 21 | 529-579 | Reproductive (0.259) | Cancer (0.444) | PSPORT1 |
| | | Hematopoietic/Immune (0.185) | Inflammation (0.370) | |
| | | Nervous (0.148) | Cell Proliferation (0.111) | |
| | | Gastrointestinal (0.111) | | |
| | | Cardiovascular (0.111) | | |
| 22 | 1239-1292 | Hematopoietic/Immune (0.333) | Cancer (0.500) | PSPORT1 |
| | | Gastrointestinal (0.250) | Inflammation (0.500) | |
| 23 | 755-796 | Hematopoietic/Immune (0.246) | Inflammation (0.457) | pINCY |
| | | Reproductive (0.246) | Cancer (0.421) | |
| | | Gastrointestinal (0.193) | | |
| | | Cardiovascular (0.123) | | |
| 24 | 69-113 | Nervous (1.000) | Cancer (1.000) | pINCY |
| 25 | 616-666 | Reproductive (0.302) | Cancer (0.476) | pINCY |
| | | Gastrointestinal (0.143) | Inflammation (0.381) | |
| | | Nervous (0.127) | Cell Proliferation (0.159) | |
| | | Hematopoietic/Immune (0.111) | | |
| 26 | 405-467 | Gastrointestinal (0.500) | Cancer (0.600) Inflammation | PSPORT1 |
| | | Nervous (0.200) | (0.420) Developmental (0.100) | |
| | | Urologic (0.200) | | |
| 27 | 2200-2253 | Reproductive (0.262) | Cancer (0.475) | pINCY |
| | | Nervous (0.220) | Inflammation (0.248) | |
| | | Gastrointestinal (0.128) | Cell Proliferation (0.213) | |
| 28 | 797-841 | Reproductive (0.277) | Cancer (0.617) | pINCY |
| | | Nervous (0.255) | Cell Proliferation (0.255) | |
| | | Cardiovascular (0.149) | Inflammation (0.213) | |
| 29 | 962-1003 | Nervous (0.235) Reproductive (0.176) | Cancer (0.500) | pINCY |
| | | Hematopoietic/Immune (0.147) | Inflammation (0.368) | |
| | | Gastointestinal (0.132) | Cell Proliferation (0.221) | |
| 30 | 555-578 | Reproductive (0.667) | Cancer (0.667) | pINCY |
| | | Urologic (0.333) | Cell Proliferation (0.667) | |
| 31 | 1154-1207 | Reproductive (0.350) | Cancer (0.475) | pINCY |
| | | Nervous (0.275) | Cell Proliferation (0.225) | |
| | | Cardiovascular (0.125) | Inflammation (0.200) | |
| | | Development (0.100) | | |
| 32 | 973-1002 | Nervous (0.314) | Cancer (0.549) | PSPORT |
| | | Reproductive (0.176) | Cell Proliferation (0.196) | |
| | | Cardiovascular (0.118) | Inflammation (0.255) | |
| 33 | 215-230 | Reproductive (0.447) | Cancer (0.638) | pINCY |
| | | Gastrointestinal (0.170) | Inflammation (0.213) | |
| | | Urologic (0.106) | Cell Proliferation (0.191) | |
| 34 | 852-896 | Hematopoietic/Immune (0.334) | Cancer (0.667) | pINCY |
| | | Gastrointestinal (0.333) | | |
| | | Musculoskeletal (0.333) | | |
| 35 | 661-705 | Nervous (0.234) | Cancer (0.404) | PSPORT1 |
| | | Reproductive (0.213) | Inflammation/Trauma (0.298) | |
| | | Gastrointestinal (0.191) | Cell Proliferation (0.106) | |
| 36 | 435-479 | Nervous (0.356) | Cancer (0.424) | pINCY |
| | | Reproductive (0.136) | Inflammation/Trauma (0.271) | |
| | | Urologic (0.119) | Cell Proliferation (0.271) | |
| 37 | 606-650 | Hematopoietic/Immune (0.500) | Inflammation/Trauma (0.375) | pINCY |
| | 1083-1127 | Cardiovascular (0.250) | Cancer (0.286) | |
| | | Gastrointestinal (0.125) | Neurological (0.143) | |
| | | Nervous (0.125) | | |
| 38 | 272-316 | Reproductive (0.300) | Cancer (0.450) | pINCY |
| | | Hematopoietic/Immune (0.186) | Inflammation/Trauma (0.393) | |
| | | Nervous (0.157) | Cell Proliferation (0.129) | |

TABLE 4

| Nucleotide | | |
|---|---|---|
| SEQ ID NO: | Library | Library Comment |
| 20 | SCORNOT01 | The library was constructed using RNA isolated from spinal cord tissue removed from |
| | | a 71-year-old Caucasian male who died from respiratory arrest. Patient history |
| | | included myocardial infarction, gangrene, and end stage renal disease. |
| 21 | BRAITUT01 | The library was constructed using RNA isolated from brain tumor tissue removed from |
| | | a 50-year-old Caucasian female during a frontal lobectomy. Pathology indicated |
| | | recurrent grade 3 oligoastrocytoma with focal necrosis and extensive calcification. |
| | | Patient history included a speech disturbance and epilepsy. Patient's brain had also |
| | | been irradiated with a total dose of 5,082. Family history included a brain tumor. |
| 22 | COLNNOT05 | The library was constructed using RNA isolated from the sigmoid colon tissue of a |
| | | 40-year-old Caucasian male during a partial colectomy. Pathology indicated Crohn's |
| | | disease involving the proximal colon and cecum. The ascending and transverse colon |
| | | displayed linear ulcerations and skip lesions. There was transmural inflammation |
| | | but no fistulas. |
| 23 | TESTTUT02 | The library was constructed using RNA isolated from testicular tumor removed from a |
| | | 31-year-old Caucasian male during unilateral orchiectomy. Pathology indicated |
| | | embryonal carcinoma. |
| 24 | BRAITUT07 | The library was constructed using RNA isolated from left frontal lobe tumor tissue |
| | | removed from the brain of a 32-year-old Caucasian male during excision of a cerebral |
| | | meningeal lesion. Pathology indicated low grade desmoplastic neuronal neoplasm. |
| | | Family history included atherosclerotic coronary artery disease. |
| 25 | PROSNOT20 | The library was constructed using RNA isolated from diseased prostate tissue removed |
| | | from a 65-year-old Caucasian male during a radical prostatectomy. Pathology |
| | | indicated adenofibromatous hyperplasia. Pathology for the associated tumor tissue |
| | | indicated an adenocarcinoma. |
| 26 | PANCNOT04 | The library was constructed using RNA isolated from the pancreatic tissue of a 5- |
| | | year-old Caucasian male who died in a motor vehicle accident. |
| 27 | BRAINOT09 | The library was constructed using RNA isolated from brain tissue removed from a |
| | | Caucasian male fetus, who died at 23 weeks' gestation. |
| 28 | LUNGNOT20 | The library was constructed using RNA isolated from right upper lobe lung tissue |
| | | removed from a 61-year-old Caucasian male. Pathology indicated panacinal emphysema. |
| | | Patient history included angina pectoris, and gastric ulcer. Family history included |
| | | a subdural hemorrhage, cancer, atherosclerotic coronary artery disease, and |
| | | pneumonia. |
| 29 | ADRENOT07 | The library was constructed using RNA isolated from adrenal tissue removed from a |
| | | 61-year-old female during a bilateral adrenalectomy. Pathology indicated no |
| | | significant abnormality of the right and left adrenal glands. Patient history |
| | | included an unspecified disorder of the adrenal glands. |
| 30 | SMCANOT01 | The library was constructed using RNA isolated from an aortic smooth muscle cell |
| | | line derived from the explanted heart of a male during a heart transplant. |
| 31 | ADRETUT05 | The library was constructed using RNA isolated from adrenal tumor tissue removed |
| | | from a 52-year-old Caucasian female during a unilateral adrenalectomy. Pathology |
| | | indicated a pheochromocytoma. |
| 32 | ADRETUT01 | The library was constructed using RNA isolated from right adrenal tumor tissue |
| | | removed from a 50-year-old Turkish male during a unilateral adrenalectomy. Pathology |
| | | indicated a metastatic renal cell carcinoma. The patient presented with |
| | | corticoadrenal insufficiency, incisional hernia, and non-alcoholic steato hepatitis. |
| | | Patient history included renal cell carcinoma. Family history included liver cancer. |
| 33 | PTHYNOT03 | The library was constructed using RNA isolated from the left parathyroid tissue of a |
| | | 69-year-old Caucasian female during a partial parathyroidectomy. Pathology |
| | | indicated hyperplasia. The patient presented with primary hyperparathyroidism. |
| 34 | CONNTUT04 | The library was constructed using RNA isolated from tumorous spinal tissue removed |
| | | from a 35-year-old Caucasian male during an exploratory laparotomy. Pathology |
| | | indicated schwannoma with degenerative changes. Patient history included anxiety, |
| | | depression, neurofibromatosis and benign neoplasm of the scrotum. Family history |
| | | included brain cancer, liver disease, and multiple sclerosis. |
| 35 | COLNNOT01 | This library was constructed using RNA isolated from colon tissue removed from a 75- |
| | | year-old Caucasian male during a hemicolectomy. |
| 36 | BRAINOT12 | This library was constructed using RNA isolated from brain tissue removed from the |
| | | right frontal lobe of a 5-year-old Caucasian male during a hemispherectomy. |
| | | Pathology indicated extensive polymicrogyria and mild to moderate gliosis |
| | | (predominantly subpial and subcortical), which are consistent with chronic seizure |
| | | disorder. Family history included a cervical neoplasm. |
| 37 | LUNGNOT23 | This library was constructed using RNA isolated from left lobe lung tissue removed |
| | | from a 58-year-old Caucasian male. Pathology for the associated tumor tissue |
| | | indicated metastatic grade 3 (of 4) osteosarcoma. Patient history included soft |
| | | tissue cancer, secondary cancer of the lung, prostate cancer, and an acute duodenal |
| | | ulcer with hemorrhage. Family history included prostate cancer, breast cancer, and |
| | | acute leukemia. |
| 38 | OVARDIT04 | This library was constructed using RNA isolated from diseased left ovary tissue |
| | | removed from a 22-year-old Caucasian female during a left ovarian cystectomy. |
| | | Pathology indicated a mature cystic teratoma (dermoid cyst) of the left ovary which |
| | | consisted of aggregate pieces of fibrous tissue and hair. Patient history included |
| | | capsular disruption of spleen, mononucleosis, and chlamydia. |

TABLE 5

| Program | Description | Reference | Parameter Threshold |
|---|---|---|---|
| ABI | A program that removes vector sequences and | Perkin-Elmer Applied Biosystems, | |
| FACTURA | masks ambiguous bases in nucleic acid sequences. | Foster City, CA. | |
| ABI/ | A Fast Data Finder useful in comparing and | Perkin-Elmer Applied Biosystems, | Mismatch <50% |
| PARACEL | annotating amino acid or nucleic acid sequences. | Foster City, CA; Paracel Inc., Pasadena, CA. | |
| FDF | | | |
| ABI | A program that assembles nucleic acid sequences. | Perkin-Elmer Applied Biosystems, | |
| AutoAssembler | | Foster City, CA. | |
| BLAST | A Basic Local Alignment Search Tool useful in | Altschul, S. F. et al. (1990) J. Mol. Biol. | ESTs: Probability value = |
| | sequence similarity search for amino acid and | 215: 403-410; Altschul, S. F. et al. (1997) | 1.0E−8 or less |
| | nucleic acid sequences. BLAST includes five | Nucleic Acids Res. 25: 3389-3402. | Full Length sequences: |
| | functions: blastp, blastn, blastx, tblastn, | | Probability value = 1.0E−10 |
| | and tblastx. | | or less |
| FASTA | A Pearson and Lipman algorithm that searches for | Pearson, W. R. and D. J. Lipman (1988) Proc. | ESTs: fasta E value = 1.06E−6 |
| | similarity between a query sequence and a group | Natl. Acad Sci. 85: 2444-2448; Pearson, W. R. | Assembled ESTs: fasta |
| | of sequences of the same type. FASTA comprises | (1990) Methods Enzymol. 183: 63-98; and | Identity = 95% or greater and |
| | as least five functions: fasta, tfasta, fastx, | Smith, T. F. and M. S. Waterman (1981) Adv. | Match length = 200 bases |
| | tfastx, and ssearch. | Appl. Math. 2: 482-489. | or greater; fastx E value = |
| | | | 1.0E−8 or less Full Length |
| | | | sequences: fastx score = |
| | | | 100 or greater |
| BLIMPS | A BLocks IMProved Searcher that matches a | Henikoff, S and J. G. Henikoff, Nucl. Acid | Score = 1000 or greater; |
| | sequence against those in BLOCKS, PRINTS, | Res., 19: 6565-72, 1991. J. G. Henikoff and | Ratio of Score/Strength = 0.75 |
| | DOMO, PRODOM, and PFAM databases to | S. Henikoff (1996) Methods Enzymol. | or larger; and, if applicable, |
| | search for gene families, sequence homology, | 266: 88-105; and Attwood, T. K. et al. | Probability value = 1.0E−3 |
| | and structural fingerprint regions. | (1997) J. Chem. Inf. Comput. Sci. 37: 417-424. | or less |
| HMMER | An algorithm for searching a query sequence | Krogh, A. et al. (1994) J. Mol. Biol., | Score = 10-50 bits for |
| | against hidden Markov model (HMM)-based | 235: 1501-1531; Sonnhammer, E. L. L. et al. | PFAM hits, depending on |
| | databases of protein family consensus sequences, | (1988) Nucleic Acids Res. 26: 320-322. | individual protein families |
| | such as PFAM. | | |
| ProfileScan | An algorithm that searches for structural and | Gribskov, M. et al. (1988) CABIOS 4: 61-66; | Normalized quality score ≧ |
| | sequence motifs in protein sequences that match | Gribskov, et al. (1989) Methods Enzymol. | GCG-specified “HIGH” value |
| | sequence patterns defined in Prosite. | 183: 146-159; Bairoch, A. et al. (1997) | for that particular Prosite motif. |
| | | Nucleic Acids Res. 25: 217-221. | Generally, score = 1.4-2.1. |
| Phred | A base-calling algorithm that examines automated | Ewing, B. et al. (1998) Genome | |
| | sequencer traces with high sensitivity and | Res. 8: 175-185; Ewing, B. and P. Green | |
| | probability. | (1998) Genome Res. 8: 186-194. | |
| Phrap | A Phils Revised Assembly Program including | Smith, T. F. and M. S. Waterman (1981) Adv. | Score = 120 or greater; |
| | SWAT and CrossMatch, programs based on | Appl. Math. 2: 482-489; Smith, T. F. and | Match length = 56 or greater |
| | efficient implementation of the Smith-Waterman | M. S. Waterman (1981) J. Mol. Biol. 147: | |
| | algorithm, useful in searching sequence homology | 195-197; and Green, P., University of | |
| | and assembling DNA sequences. | Washington, Seattle, WA. | |
| Consed | A graphical tool for viewing and editing Phrap | Gordon, D. et al. (1998) Genome | |
| | assemblies | Res. 8: 195-202. | |
| SPScan | A weight matrix analysis program that scans | Nielson, H. et al. (1997) Protein Engineering | Score = 3.5 or greater |
| | protein sequences for the presence of secretory | 10: 1-6; Claverie, J. M. and S. Audic (1997) | |
| | signal peptides. | CABIOS 12: 431-439. | |
| Motifs | A program that searches amino acid sequences for | Bairoch et al. supra; Wisconsin | |
| | patterns that matched those defined in Prosite. | Package Program Manual, version | |
| | | 9, page M51-59, Genetics Computer | |
| | | Group, Madison, WI. | |

1-20. (canceled)

21. An isolated polypeptide selected from the group consisting of:
(a) a polypeptide comprising the amino acid sequence of SEQ ID NO:5, 6, 7, 10, 11, 12, 17, or 19;
(b) a polypeptide comprising an amino acid sequence at least 90% identical to the amino acid sequence of SEQ ID NO:5, 6, 7, 10, 11, 12, 17, or 19;
(c) a biologically active fragment of a polypeptide having the amino acid sequence of SEQ ID NO:5, 6, 7, 10, 11, 12, 17, or 19; and
(d) an immunogenic fragment of a polypeptide having the amino acid sequence of SEQ ID NOS:5, 6, 7, 10, 11, 12, 17, or 19.

22. An isolated polypeptide of claim 21 selected from the group consisting of SEQ ID NO:5, 6, 7, 10, 11, 12, 17, and 19.

23. An isolated polynucleotide encoding the polypeptide of claim 21.

24. An isolated polynucleotide encoding the polypeptide of claim 22.

25. An isolated polynucleotide of claim 24 selected from the group consisting of SEQ ID NO:24, 25, 26, 29, 30, 31, 36, and 38.

26. A recombinant polynucleotide comprising a promoter sequence operably linked to a polynucleotide of claim 23.

27. A cell transformed with a recombinant polynucleotide of claim 26.

28. A pharmaceutical composition comprising the polypeptide of claim 21 in conjunction with a suitable pharmaceutical carrier.

29. A method for producing a polypeptide of claim 21, the method comprising:
culturing a cell under conditions suitable for expression of the polypeptide, wherein said cell is transformed with a recombinant polynucleotide, and said recombinant polynucleotide comprises a promoter sequence operably linked to a polynucleotide encoding a polypeptide of claim 21, and
recovering the polypeptide so expressed.

30. An isolated polynucleotide selected from the group consisting of:
(a) a polynucleotide comprising the polynucleotide sequence of SEQ ID NO:24, 25, 26, 29, 30, 31, 36, or 38,
(b) a polynucleotide comprising a polynucleotide sequence at least 90% identical to the polynucleotide sequence of SEQ ID NO:24, 25, 26, 29, 30, 31, 36, or 38,
(c) a polynucleotide complementary to the polynucleotide of (a);
(d) a polynucleotide complementary to the polynucleotide of (b); and
(e) an RNA equivalent of (a)-(d).

31. A method for detecting a target polynucleotide in a sample, said target polynucleotide having a sequence of a polynucleotide of claim 30, the method comprising:
hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof; and
detecting the presence or absence of said hybridization complex and, optionally, if present, the amount thereof.

32. A method for detecting a target polynucleotide in a sample, said target polynucleotide having a sequence of a polynucleotide of claim 30, the method comprising:
amplifying said target polynucleotide or fragment thereof using polymerase chain reaction; and
detecting the presence or absence of said target polynucleotide and, optionally, if present, the amount thereof.

33. An isolated antibody which specifically binds to a polypeptide of claim 21.

34. A method for treating or preventing a disease or condition associated with decreased expression of functional VEAS, the method comprising administering to a subject in need of such treatment an effective amount of the pharmaceutical composition of claim 28.

35. The isolated polypeptide of claim 21, wherein said polypeptide comprises an amino acid sequence at least 95% identical to the amino acid sequence of SEQ ID NO:5, 6, 7, 10, 11, 12, 17, or 19.

36. The isolated polynucleotide of claim 30, wherein said polynucleotide comprises a polynucleotide sequence at least 95% identical to the polynucleotide sequence of SEQ ID NO:5, 6, 7, 10, 11, 12, 17, or 19.


SEQUENCE LISTING (38 sequences)

SEQ ID NO: 1 - protein, 144 aa, Homo sapiens
  misc_feature: Incyte ID No 665637CD1
MIKFFLMVNKQGQTRLSKYYEHVDINKRTLLETEVIKSCLSRSNEQCSFIEYKDFKLIYRQYAALFIVVGVNDTENEMAIYEFIHNFVEVLDEYFSRVSELDIMFNLDKVHIILDEMVLNGCIVETNRARILAPLLILDKMSES

SEQ ID NO: 2 - protein, 177 aa, Homo sapiens
  misc_feature: Incyte ID No 745823CD1
MGNIFEKLFKSLLGKKKMRILILSLDTAGKTTILYKLKLGETVPAVPTVGFCVETVEYKNNTFAVWDVGSHFKIRPLWQHFFQNTKGARSPGSTHQGSLASGVLPIKCSHVEFGMWKGGRSHPFLPHSSRCAGSGGQLDSILPHQSPAWGPWGCKDLSSGFPSFLTSSILWKSAVVK

SEQ ID NO: 3 - protein, 408 aa, Homo sapiens
  misc_feature: Incyte ID No 776854CD1
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLRDYKEALMIPAKDGVKLPYFFHAGETDPKEDLGGGCSHGGRDQEGSSLQHPTCHPRKTKAGWREWWAVSFPTQA

SEQ ID NO: 4 - protein, 553 aa, Homo sapiens
  misc_feature: Incyte ID No 1273556CD1
MTRGGPGGRPGLPQPPPLLLLLLLLPLLLVTAEPPKPAGVYYATAYWMPAEKTVQVKNVMDKNGDAYGFYNNSVKTTGWGILEIRAGYGSQTLSNEIIMFVAGFLEGYLTAPHMNDHYTNLYPQLITKPSIMDKVQDFMEKQDKWTRKNIKEYKTDSFWRHTGYVMAQIDGLYVGAKKRAILEGTKPMTLFQIQFLNSVGDLLDLIPSLSPTKNGSLKVFKRWDMGHCSALIKVLPGFENILFAHSSWYTYAAMLRIYKHWDFNIIDKDTSSSRLSFSSYPGFLESLDDFYILSSGLILLQTTNSVFNKTLLKQVIPETLLSWQRVRVANMMADSGKRWADIFSKYNSGTYNNQYMVLDLKKVKLNHSLDKGTLYIVEQIPTYVEYSEQTDVLRKGYWPSYNVPFHEKIYNWSGYPLLVQKLGLDYSYDLAPRAKIFRRDQGKVTDTASMKYIMRYNNYKKDPYSRGDPCNTICCREDLNSPNPSPGGCYDTKVADIYLASQYTSYAISGPTVQGGLPVFRWDRFNKTLHQGMPEVYNFDFITMKPILKLDIK

SEQ ID NO: 5 - protein, 179 aa, Homo sapiens
  misc_feature: Incyte ID No 1505808CD1
MDPAGPSAPRRWTARSPGRKNTRSRPYLRVSSDPWRSHCGSRAIAMEFVMKQALGGATKDMGKMLGGDEEKDPDAAKKEEERQEALRQAEEERKAKYAKMEAEREAVRQGIRDKYGIKKKEEREAEAQAAMEANSEGSLTRPKKAIPPGCGDEVEEEDESILDTVIKYLPGPLQDMLKK

SEQ ID NO: 6 - protein, 336 aa, Homo sapiens
  misc_feature: Incyte ID No 1814911CD1
MEKLRLLGLRYQEYVTRHPAATAQLETAVRGFSYLLAGRFADSHELSELVYSASNLLVLLNDGILRKELRKKLPVSLSQQKLLTWLSVLECVEVFMEMGAAKVWGEVGRWLVIALIQLAKAVLRMLLLLWFKAGLQTSPPIVPLDRETQAQPPDGDHSPGNHEQSYVGKRSNRVVRTLQNTPSLHSRHWGAPQQREGRQQQHHEELSATPTPLGLQETIAEFLYIARPLLHLLSLGLWGQRSWKPWLLAGVVDVTSLSLLSDRKGLTRRERRELRRRTILLLYYLLRSPFYDRFSEARILFLLQLLADHVPGVGLVTRPLMDYLPTWQKIYFYSWG

SEQ ID NO: 7 - protein, 240 aa, Homo sapiens
  misc_feature: Incyte ID No 2087812CD1
MSPLLFGAGLVVLNLVTSARSQKTEPLSGSGDQPLFRGADRYDFAIMIPPGGTECFWQFAHQTGYFYFSYEVQRTVGMSHDRHVAATAHNPQGFLIDTSQGVRGQINFSTQETGFYQLCLSNQHNHFGSVQVYLNFGVFYEGPETDHKQKERKQLNDTLDAIEDGTQKVQNNIFHMWRYYNFARMRKMADFFLIQSNYNYVNWWSTAQSLVIILSGILQLYFLKRLFNVPTTTDTKKPRC

SEQ ID NO: 8 - protein, 955 aa, Homo sapiens
  misc_feature: Incyte ID No 2149274CD1
MPAVSKGDGMRGLAVFISDIRNCKSKEAEIKRINKELANIRSKFKGDKALDGYSKKKYVCKLLFIFLLGHDIDFGHMEAVNLLSSNKYTEKQIGYLFISVLVNSNSELIRLINNAIKNDLASRNPTFMCLALHCIANVGSREMGEAFAADIPRILVAGDSMDSVKQSAALCLLRLYKASPDLVPMGEWTARVVHLLNDQHMGVVTAAVSLITCLCKKNPDDFKTCVSLAVSRLSRIVSSDSTELQDYTYYFVPAPWLSVKLLRLLQCYPPPEDAAVKGRLVECLETVLNKAQEPPKSKKVQHSNAKNAILFETISLIIHYDSEPNLLVRACNQLGQFLQHRETNLRYLALESMCTLASSEFSHEAVKTHIDTVINALKTERDVSVRQRAADLLYAMCDRSNAKQIVSEMLRYLETADYAIREEIVLKVAILAEKYAVDYSWYVDTILNLIRIACDYVSEEVWYRVLQIVTNRDDVQGYAAKTVFEALQAPACHENMVKVGGYILGEFGNLIAGDPRSSPPVQFSLLHSKFHLCSVATRALLLSTYIKFINLFPETKATIQGVLRAGSQLRNADVELQQRAVEYLTLSSVASTDVLATVLEEMPPFPERESSILAKLKRKKGPGAGSALDDGRRDPSSNDINGGMEPTPSTVSTPSPSADLLGLRAAPPPAAPPASAGAGNLLVDVFDGPAAQPSLGPTPEEAFLSPGPEDIGPPIPEADELLNKFVCKNNGVLFENQLLQIGVKSEFRQNLGRMYLFYGNKTSVQFQNFSPTVVHPGDLQTQLAVQTKRVAAQVDGGAQVQQVLNIECLRDFLTPPLLSVRFRYGGAPQALTLKLPVTINKFFQPTEMAAQDFFQRWKQLSLPQQEAQKIFKANHPMDAEVTKAKLLGFGSALLDNVDPNPENFVGAGIIQTKALQVGCLLRLEPNAQAQMYRLTLRTSKEPVSRHLCELLAQQF

SEQ ID NO: 9 - protein, 247 aa, Homo sapiens
  misc_feature: Incyte ID No 2355124CD1
MLVLRSGLTKALASRTLAPQVCSSFATGPRQYDGTFYEFRTYYLKPSNMNAFMENLKKNIHLRTSYSELVGFWSVEFGGRTNKVFHIWKYDNFAHRTEVQKALAKDKEWQEQFLIPNLALIDKQESEITYLVPWCKLEKPPKEGVYELATFQMKPGGPALWGDAFKRAVHAHVNLGYTKLVGVFHTEYGALNRVHVLWWNESADSRAAGRHKSHEDPRVVAAVRESVNYLVSQQNMLLIPTSFSPLK

SEQ ID NO: 10 - protein, 532 aa, Homo sapiens [sequence omitted]

SEQ ID NO: 11 - protein, 154 aa, Homo sapiens
  misc_feature: Incyte ID No 2483906CD1
MIHFILLFSRQGKLRLQKWYITLPDKERKKITREIVQIILSRGHRTSSFVDWKELKLVYKRYASLYFCCAIENQDNELLTLEIVHRYVELLDKYFGNVCELDIIFNFEKAYFILDEFIIGGEIQETSKKIAVKAIEDSDMLQETMEEYMNKPTF

SEQ ID NO: 12 - protein, 684 aa, Homo sapiens
  misc_feature: Incyte ID No 2499488CD1
MWHETSNRIPSKRTVSFVLSTGLYPISFQVHLVRDGVLTRFSVATEYQKNAQIEKLRSEIVVLKEELQLTRSELEAAHHASAVRFSKEYEMQKTKEEDFLKLFDRWKEEEKEKLVDEMEKVKEMFMKEFKELTSKNSALEYQLSEIQKSNMQIKSNIGTLKDAHEFKEDRSPYPQDFHNVMQLLDSQESKWTARVQAIHQEHKKEKGRLLSHIEKLRTSMIDDLNASNVFYKKRIEELGQRLQEQNELIITQRQQIKDFTCNPLNSISEPKGNPLAWQAFESQPAAPAVPMNAPALHTLETKSSLPMVHEQAFSSHILEPIEELSEEEKGRENEQKLNNNKMHLRKALKSNSSLTKGLRTMVEQNLMEKLETLGINADIRGISSDQLHRVLKSVESERHKQEREIPNFHQIREFLEHQVSCKIEEKALLSSDQCSVSQMDTLSTGEVPKMIQLPSKNRQLIRQKAVSTDRTSVPKIKKNVMEDPFPRKSSTITTPPFSSEEEQEDDDLIRAYASPGPLPVPPPQNKGSFGKNTVKSDADGTEGSEIEDTDDSPKPAGVAVKTPTEKVEKMFPHRKNVNKPVGGTNVPEMFIKKEELQELKCADVEDEDWDISSLEEEISLGKKSGKEQKEPPPAKNEPHFAHVLNAWGAFNPKGPKGEGLQENESSTLKSSLVTVTDWSDTSDV

SEQ ID NO: 13 - protein, 576 aa, Homo sapiens
  misc_feature: Incyte ID No 2559148CD1
MSTSSLRRQMKNIVHNYSEAEIKVREATSNDPWGPSSSLMSEIADLTYNVVAFSEIMSMIWKRLNDHGKNWRHVYKAMTLMEYLIKTGSERVSQQCKENMYAVQTLKDFQYVDRDGKDQGVNVREKAKQLVALLRDEDRLREERAHALKTKEKLAQTATASSAAVGSGPPPEAEQAWPQSSGEEELQLQLALAMSKEEADQPPSCGPEDDAQLQLALSLSREEHDKEERIRRGDDLRLQMAIEESKRETGGKEESSLMDLADVFTAPAPAPTTDPWGGPAPMAAAVPTAAPTSDPWGGPPVPPAADPWGGPAPTPASGDPWRPAAPAGPSVDPWGGTPAPAAGEGPTPDPWGSSDGGVPVSGPSASDPWTPAPAFSDPWGGSPAKPSTNGTTAAGGFDTEPDEFSDFDRLRTALPTSGSSAGELELLAGEVPARSPGAFDMSGVRGSLAEAVGSPPPAATPTPTPPTRKTPESFLGPNAALVDLDSLVSRPGPTPPGAKASNPFLPGGGPATGPSVTNPFQPAPPATLTLNQLRLSPVPPVPGAPPTYISPLGGGPGLPPMMPPGPPAPNTNPFLL

SEQ ID NO: 14 - protein, 425 aa, Homo sapiens
  misc_feature: Incyte ID No 3321481CD1
MSASAVFILDVKGKPLISRNYKGDVAMSKIEHFMPLLVQREEEGALAPLLSHGQVHFLWIKHSNLYLVATTSKNANASLVYSFLYKTIEVFCEYFKELEEESIRDNFVIVYELLDELMDFGFPQTTDSKILQEYITQQSNKLETGKSRVPPTVTNAVSWRSEGIKYKKNEVFIDVIESVNLLVNANGSVLLSEIVGTIKLKVFLSGMPELRLGLNDRVLFELTGLSGSKNKSVELEDVKFHQCVRLSRFDNDRTISFIPPDGDFELMSYRLSTQVKPLIWIESVIEKFSHSRVEIMVKAKGQFKKQSVANGVEISVPVPSDADSPRFKTSVGSAKYVPERNVVIWSIKSFPGGKEYLMRAHFGLPSVEKEEVEGRPPIGVKFEIPYFTVSGIQVRYMKIIEKSGYQALPWVRYITQSGDYQLRTS

SEQ ID NO: 15 - protein, 167 aa, Homo sapiens [sequence omitted]

SEQ ID NO: 16 - protein, 739 aa, Homo sapiens
  misc_feature: Incyte ID No 1227327CD1
MPYLGSEDVVKELKKALCNPHIQADRLRYRNVIQRVIRYMTQGLDMSGVFMEMVKASATVDIVQKKLVYLYMCTYAPLKPDLALLAINTLCKDCSDPNPMVRGLALRSMCSLRMPGVQEYIQQPILNGLRDKASYVRRVAVLGCAKMHNLHGDSEVDGALVNELYSLLRDQDPIVVVNCLRSLEEILKQEGGVVINKPIAHHLLNRMSKLDQWGQAEVLNFLLRYQPRSEEELFDILNLLDSFLKSSSPGVVMGATKLFLILAKMFPHVQTDVLVRVKGPLLAACSSESRELCFVALCHVRQILHSLPGHFSSHYKKFFCSYSEPHYIKLQKVEVLCELVNDENVQQVLEELRGYCTDVSADFAQAAIFAIGGIARTYTDQCVQILTELLGLRQEHITTVVVQTFRDLVWLCPQCTEAVCQALPGCEENIQDSEGKQALIWLLGVHGERIPNAPYVLEDFVENVKSETFPAVKMELLTALLRLFLSRPAECQDMLGRLLYYCIEEEKDMAVRDRGLFYYRLLLVGIDEVKRILCSPKSDPTLGLLEDPAERPVNSWASDFNTLVPVYGKAHWATISKCQGAERCDPELPKTSSFAASGPLIPEENKERVQELPDSGALMLVPNRQLTADYFEKTWLSLKVAHQQVLPWRGEFHPDTLQMALQVVNIQTIAMSRAGSRPWKAYLSAQDDTGCLFLTELLLEPGNSEMQISVKQNEARTETLNSFISVLETVIGTIEEIKS

SEQ ID NO: 17 - protein, 742 aa, Homo sapiens [sequence omitted]

SEQ ID NO: 18 - protein, 325 aa, Homo sapiens
  misc_feature: Incyte ID No 2611704CD1
MAHFVQGTSRMIAAESSTEHKECAEPSTRKNLMNSLEQKIRCLEKQRKELLEVNQQWDQQFRSMKELYERKVAELKTKLDAAERFLSTREKDPHQRQRKDDRQREDDRQRDLTRDRLQREEKEKERLNEELHELKEENKLLKGKNTLANKEKEHYECEIKRLNKALQDALNIKCSFSEDCLRKSRVEFCHEEMRTEMEVLKQQVQIYEEDFKKERSDRERLNQEKEELQQINETSQSQLNRLNSQIKACQMEKEKLEKQLKQMYCPPCNCGLVFHLQDPWVPTGPGAVQKQREHPPDYQWYALDQLPPDVQHKANGLSSVKKVHP

SEQ ID NO: 19 - protein, 299 aa, Homo sapiens
  misc_feature: Incyte ID No 5136672CD1
MEHFDASLSTYFKALLGPRDTRVKGWFLLDNYIPTFICSVIYLLIVWLGPKYMRNKQPFSCRGILVVYNLGLTLLSLYMFCELVTGVWEGKYNFFCQGTRTAGESDMKIIRVLWWYYFSKLIEFMDTFFFILRKNNHQITVLHVYHHASMLNIWWFVMNWVPCGHSYFGATLNSFIHVLMYSYYGLSSVPSMRPYLWWKKYITQGQLLQFVLTIIQTSCGVIWPCTFPLGWLYFQIGYMISLIALFTNFYIQTYNKKGASRRKDHLKDHQNGSMAAVNGHTNSFSPLENNVKPRKLRKD

SEQ ID NO: 20 - DNA, 1050 bp, Homo sapiens
  misc_feature: Incyte ID No 665637CB1
aagccaaaatggctgccccgaggaggcccgcaccgcgtagccagtgaaggttggggagcaagcttatgcgggaaagagggagggggactccaggaaaagccgttgagaggaccatcacaacctgagcagcacaggttccagttacagccatcccttgtcataacttttgaactgtatttggaaaaatactggccaggaaagaaaaatgataaaatttttcctcatggtgaataaacaagggcagactcgactttctaagtactatgaacatgtggatattaataagcgtacacttctggaaacagaagtcataaagagctgtctctctcgatccaatgaacaatgctctttcattgaatataaggattttaagctgatatatcggcagtatgcagctctcttcattgtggttggagttaatgacactgagaacgagatggctatttatgaattcattcataactttgtggaagttttagatgagtatttcagccgagtgagtgaattagatataatgtttaatttggataaagtacacatcattttggatgagatggtgttaaatggctgcattgtggaaactaacagggcaagaattcttgcccctctactaattcttgataagatgtcagaaagctgaaaggaagtctcttcgagacaatatggatttatcagaaatgcgagtaccgtggaatacatctcaacatgttaacccagaagaatctggaagaccacaattacaaaatggggtatccttccaaagacattataaataggcattttccacagttcctaaaaagaaaacacaactgtactttaaaatatgtacaaagaaaaaaatttctttaaactgagagagaagttttattttctaattgtaaacatatctgtcgcactttaaattctgttgagcacctaaggaacccttcttggtctatgcttcttttgcaaattgaattcaggaatagcaggatggtagtgggaagaaagtatggcagttttccgtcagccaataaagttttaaaatttaaacacaaaaaaaaaaaaaaa

SEQ ID NO: 21 - DNA, 916 bp, Homo sapiens
  misc_feature: Incyte ID No 745823CB1
gaacatggtattgccagagcttcatggcgtcagctacggcgctccctccctcccaggcccttactcttctttgcgttggccagatatgtcgtcactcacaaaacttcgagctcattggtgcaaaagctccaggaggcggagggtgattggttggttgctgttcctgcccccacgcatgtcgtggtttagctgaactgagctgaaatcctaaaggccgcggagtcggcggtgttgtaggtagcggtaccttgagtggcaacagaattcgattaaattacaatgggaaacatttttgaaaagctctttaaaagtctacttgggaaaaaaaagatgcggattcttatattgagtttggatacagctggaaaaaccaccatcttgtataaattgaagctgggggagactgtgcctgccgtccctacagtaggtttctgtgtggagacagtagaatataaaaataacaccttcgctgtctgggatgttggcagccacttcaaaatcagacctctgtggcagcattttttccagaacacaaaaggtgccagaagcccaggaagcacacatcaaggctcacttgccagcggggtgctgccaataaaatgtagtcacgtggaatttggaatgtggaaaggaggtagaagtcatcctttcctcccccatagcagcaggtgtgcaggctctggtggtcagctggactccatactcccccaccagtcaccagcctggggaccgtggggctgcaaggacctcagcagcggtttcccaagtttcctgacttcttccatcctctggaaatcagctgtggtaaagtagcctgaaagccagtggtgcaaccccatccccacaaccttcaccacctctagcacctccagtgataagcactaattgcctatatacaacccttttttgtttgaaa

SEQ ID NO: 22 - DNA, 1930 bp, Homo sapiens
  misc_feature: Incyte ID No 776854CB1
cttttccggtgctctgcacagatgctggggcactgagcaaacagccctcagtttctggagctgttccgagtcccgtggagtctccatctgagccctttcctagtccaggcatcccgatgttggtggatggcccatctgagcggccagccctgtgcttcttgctgttggctgtggcaatgtctttcttcggctcagctctatccatagatgaaacacgggcgcatctgttgttgaaagaaaagatgatgcggctcggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaggctgctgccggtgtatgagctcagtggagagcaccatgacgaagagtggtcagtgaagacttaccaggaagtagctcagaagtttgtggaaactcaccctgagtttattggaatcaaaatcatttattcggatcacagatccaaagatgtggctgtcatcgcagaatccatccgaatggccatggggctccgaatcaagttccccacggtggtggcagggtttgacctggtggggcatgaggacactggccactccttgcgtgactacaaggaagctctgatgatccccgccaaggatggcgttaagctgccttacttcttccacgccggagaaacagatccaaaggaagatttgggaggaggctgttcacatggaggccgggaccaggagggcagctctcttcagcatcccacctgtcaccctcggaagaccaaggcggggtggagagagtggtgggcagtttcatttcccacccaggcttaagctctgctctatcacaccttctctcagagaaagggaccttgtgcattaatctctttttcctgacatttcgagtcaatttagatccaatttagatcaaagtctgctctaggagagcagaagaattgtttgagggggagggggttgggcgagggtgttgttaaccttgaagagcttgtggtctggttggggaaaatcatacttatgtgtgtgaaataattctggccctacaaaacattgcagcacaggacctaacaggttccaagatgtatggtccttggagatgattttgccaggacttgggtatctaatacctaaaagtaatgtccttatttttatttttttattttattttatgttattttttgagacggagtctcgctctgtactccagcctggcgacagagcaagactgtgtctcaaaaaacaacaacaacaacaacaaaaaactcggttttgctacttactgtgtgaacatccttggtcaatttatagaagctattaagtttcaactttctcattagaatgataaattaataaattagatgattaatttaataaagccatatcatacctctctttagcattga

SEQ ID NO: 23 - DNA, 1809 bp, Homo sapiens
  misc_feature: Incyte ID No 1273556CB1
cgcagaggaaggtcgcggccgtggagcgatgacccgcggcggtccgggcgggcgcccggggctgccacagccgccgccgcttctgctgctgctgctgctgctgccgctgttgttagtcaccgcggagccgccgaaacctgcaggagtctactatgcaactgcatactggatgcctgctgaaaagacagtacaagtcaaaaatgtaatggacaagaatggggacgcctatggcttttacaataactctgtgaaaaccacaggctggggcatcctggagatcagagctggctatggctctcaaaccctgagcaatgagatcatcatgtttgtggctggctttttggagggttacctcactgccccacacatgaatgaccactacacaaacctctacccacagctgatcacgaaaccttccatcatggataaagtgcaggattttatggagaagcaagataagtggacccggaaaaatatcaaagaatacaagactgattcattttggagacatacaggctatgtgatggcacaaatagatggcctctatgtaggagcaaagaagagggctattttagaagggacaaagccaatgaccctgttccagattcagttcctgaatagtgttggagatctattggatctgattccctcactctctcccacaaaaaacggcagcctaaaggtttttaagagatgggacatgggacattgctccgctcttatcaaggttcttcctggatttgagaacatcctttttgctcactcaagctggtacacgtatgcagccatgctcaggatatataaacactgggacttcaacatcatagataaagataccagcagtagtcgcctctctttcagcagttacccagggtttttggagtctctggatgatttttacattcttagcagtggattgatattgctgcagaccacaaacagtgtgtttaataaaaccctgctaaagcaggtaatacccgagactctcctgtcctggcaaagagtccgtgtggccaatatgatggcagatagtggcaagaggtgggcagacatcttttcaaaatacaactctggcacctataacaatcaatacatggttctggacctgaagaaagtaaagctgaaccacagtcttgacaaaggcactctgtacattgtggagcaaattcctacatatgtagaatattctgaacaaactgatgttctacggaaaggatattggccctcctacaatgttcctttccatgaaaaaatctacaactggagtggctatccactgttagttcagaagctgggcttggactactcttatgatttagctccacgagccaaaattttccggcgtgaccaagggaaagtgactgatacggcatccatgaaatatatcatgcgatacaacaattataagaaggatccttacagtagaggtgacccctgtaataccatctgctgccgtgaggacctgaactcacctaacccaagtcctggaggttgttatgacacaaaggtggcagatatctacctagcatctcagtacacatcctatgccataagtggtcccacagtacaaggtggcctccctgtttttcgctgggaccgtttcaacaaaactctacatcagggcatgccagaggtctacaactttgattttattaccatgaaaccaattttgaaacttgatataaaatgaaggagggagatgacggactagaagactgtaaataagataccaaaggcactattttagctatgtttttcccatcagaattatgcaataaaatatattaatttgtcaaaaaaaaaaaaaaa

SEQ ID NO: 24 - DNA, 638 bp, Homo sapiens
  misc_feature: Incyte ID No 1505808CB1
tcaccgggcgccacatggacccagccggcccgagcgcgccccgccgctggaccgcccgctccccgggaaggaagaacactcgctcccggccatacttgcgtgtgagttctgacccctggaggagccactgtggaagcagagcaatcgccatggagtttgtgatgaagcaggctctaggaggggccaccaaggacatggggaagatgctggggggtgacgaggagaaggacccagacgccgccaagaaggaggaggagcggcaggaggcgctgcgccaggcggaggaggagcgcaaggccaagtacgccaagatggaggcggagcgcgaggccgtgcgccagggcatccgagacaagtacggcatcaagaagaaggaggagcgcgaggccgaggcccaggccgccatggaggccaactccgaggggagcttgacgcggcccaagaaggccatcccgccgggctgcggggacgaggtggaggaggaggacgagagcatcctggacaccgtcatcaagtacctgcccgggccgctgcaggacatgctcaagaagtagccccgcgcgggacagcggccccgcggagcccccgcccctccccctacagatcctccgcggaggcccctgagggacgagcagagc

SEQ ID NO: 25 - DNA, 1477 bp, Homo sapiens
  misc_feature: Incyte ID No 1814911CB1
gcggttcaagagatggggtgaagggtggttcaccggttcttcaagtcctcagccttctggcccgcggaagttaagcaaccaagaggcgggcctaagaccggaagcaggaaggagggcgcaggaagcagggcgccgcagcctgtcgtacggtccttctgtgggtctgtcggtgccgagggcaggatggagaagctgcggctcctgggcctccgctaccaggagtacgtgactcgtcacccggccgccacggcccagctggagacagcagtgcggggcttcagttacctgctggcaggtcgattcgccgattcgcacgagctgtcagagctggtgtactctgcctctaacctgcttgtgctgctcaatgacgggatcctacggaaggagcttcggaaaaagttgcctgtgtcgctgtcccagcagaagctgctgacatggctgagcgtgctggagtgcgtggaggtgttcatggagatgggagctgccaaggtgtggggtgaagtgggccgctggcttgtcatcgccctcatccagctggccaaggctgtactgcggatgctcctgctgctctggttcaaggctggcctccagacttcaccccctatcgttccactggacagagagacccaggcacagcccccggatggtgaccacagccctggcaaccatgagcagtcctacgtggggaagcggtcaaaccgggtggtgcgaaccctccagaacacgccgtccctgcactccaggcactggggagctccccagcagcgggagggacggcagcagcagcatcacgaggagctgagtgcgacccccacccccctggggctgcaggagaccatcgcagagtttttgtacattgcccggccgctgctgcacttgctcagcctgggcctgtggggtcagaggtcgtggaaaccctggctcttggctggtgttgtggacgtgaccagcctgagcctcctgagtgacagaaagggcctgacccggagggagcggcgggagctgcggcgccggaccatcctgctgctctactacctgctgcgctctcctttctacgaccgcttctccgaggccaggatcctcttcctgctccagttgctggccgaccacgtccctggcgttggcctggtcacaaggccgctcatggattacttgcccacctggcagaaaatctacttctacagttggggctgacagacctcccggaaggagggtgtggggaggggtggggcagggagcccctcttccctaataaaactgactccggcagcgtcccagcgtgcggcctctccgtgcctaccggccaggccccacacacagccctggtcgccaccagcgttcctcccaggacacccttgactgcgctctcctgtgacgatgccactgcagcccgcaccttgtcactgctgggccaagaagccttcactaggagtgggatccaggctcctctcccacagaaagcggtgacttcacctca

SEQ ID NO: 26 - DNA, 1137 bp, Homo sapiens
  misc_feature: Incyte ID No 2087812CB1
ctccagaaagcggcatgtcccctttgctctttggggctgggctggtcgttctgaatctagtgacgtctgccaggagccagaagacagaacctctaagtggctctggggaccagccactcttccgtggagctgatcgatatgactttgccatcatgatacctccaggaggcacggaatgcttttggcaatttgcccaccagactggatacttctatttcagttacgaggttcagcggacagtggggatgtcacatgaccggcatgttgctgccacggcacataacccacagggatttctcatagacacctcccagggtgttcggggccagattaacttctctacccaagagacaggtttttatcagctttgtctaagtaatcagcataatcacttcggttctgtgcaagtgtacctcaactttggggtcttctatgaggggcctgagactgatcacaaacagaaggaaagaaaacaactgaatgatactctggatgcaattgaggacggcacacaaaaggtgcagaacaatatctttcacatgtggcgatactacaactttgcccggatgaggaaaatggctgactttttccttatccaatcaaactataactacgtgaactggtggtcgacagcccagagccttgttattattctttctgggatcctgcaactgtatttcttgaagcgtctcttcaatgttccaacaactacagatacaaagaagccaagatgctaagctaaggtgactatagcaccctggctgttttcttctggggcttagtcgaatcagctttgtaatgttatgggacaaaaatcaattatctcattaatgttttagtctgctgcacacatctaaaaaagcaaaatggcaataaaatcataacagtgaaaaagttctgaagaagtatgtattaaatgtataaaggaaaatactgttcatcatatagcagtcccttagccttcactgagtctacttagaggctaacagaaaaccttagaaggaagatgagttgaaaaaggccacaatcaagtttgcttacatataaaagcaggagttcgtaatgttttattcttaaagacttcacctctggaacttaaaataagcaatatattcagtaaagaaatagttgaattta

SEQ ID NO: 27 - DNA, 3343 bp, Homo sapiens
  misc_feature: Incyte ID No 2149274CB1
cggctcggctccttggcgctgcctggggtcctttccgcccggtccccgcttgccagcccccgctgctctgtgccctgtccggccaggcctggagccgacaccaccgccatcatgccggccgtgtccaagggcgatgggatgcgggggctcgcggtgttcatctccgacatccggaactgtaagagcaaagaggcggaaattaagagaatcaacaaggaactggccaacatccgctccaagttcaaaggagacaaagccttggatggctacagtaagaaaaaatatgtgtgtaaactgcttttcatcttcctgcttggccatgacattgactttgggcacatggaggctgtgaatctgttgagttccaataaatacacagagaagcaaataggttacctgttcatttctgtgctggtgaactcgaactcggagctgatccgcctcatcaacaacgccatcaagaatgacctggccagccgcaaccccaccttcatgtgcctggccctgcactgcatcgccaacgtgggcagccgggagatgggcgaggcctttgccgctgacatcccccgcatcctggtggccggggacagcatggacagtgtcaagcagagtgcggccctgtgcctccttcgactgtacaaggcctcgcctgacctggtgcccatgggcgagtggacggcgcgtgtggtacacctgctcaatgaccagcacatgggtgtggtcacggccgccgtcagcctcatcacctgtctctgcaagaagaacccagatgacttcaagacgtgcgtctctctggctgtgtcgcgcctgagccggatcgtctcctctgactccaccgaactccaggactacacctactacttcgtcccagcaccctggctctcggtgaagctcctgcggctgctgcagtgctacccgcctccagaggatgcggctgtaaaggggcggctggtggaatgtctggagactgtgctcaacaaggcccaggagccccccaaatccaagaaggtgcagcattccaacgccaagaacgccatcctcttcgagaccatcagcctcatcatccactatgacagtgagcccaacctcctggttcgggcctgcaaccagctgggccagttcctgcagcaccgggagaccaacctgcgctacctggccctggagagcatgtgcacgctggccagctccgagttctcccatgaagccgtcaagacgcacattgacaccgtcatcaatgccctcaagacggagcgggacgtcagcgtgcggcagcgggcggctgacctcctctacgccatgtgtgaccggagcaatgccaagcagatcgtgtcggagatgctgcggtacctggagacggcagactacgccatccgcgaggagatcgtcctgaaggtggccatcctggccgagaagtacgccgtggactacagctggtacgtggacaccatcctcaacctcatccgcattgcgtgcgactacgtgagtgaggaggtgtggtaccgtgtgctacagatcgtcaccaaccgtgatgacgtccagggctatgccgccaagaccgtctttgaggcgctccaggcccctgcctgtcacgagaacatggtgaaggttggcggctacatccttggggagtttgggaacctgattgctggggacccccgctccagccccccagtgcagttctccctgctccactccaagttccatctgtgcagcgtggccacgcgggcgctgctgctgtccacctacatcaagttcatcaacctcttccccgagaccaaggccaccatccagggcgtcctgcgggccggctcccagctgcgcaatgctgacgtggagctgcagcagcgagccgtggagtacctcaccctcagctcagtggccagcaccgacgtcctggccacggtgctggaggagatgccgcccttccccgagcgcgagtcgtccatcctggccaagctgaaacgcaagaaggggccaggggccggcagcgccctggacgatggccggagggaccccagcagcaacgacatcaacgggggcatggagcccacccccagcactgtgtcgacgccctcgccctccgccgacctcctggggctgcgggcagcccctcccccggcagcacccccggcttctgcaggagcagggaaccttctggtggacgtcttcgatggcccggccgcccagcccagcctggggcccacccccgaggaggccttcctcagcccaggtcctgaggacatcggccctcccattccggaagccgatgagttgctgaataagtttgtgtgtaagaacaacggggtcctgttcgagaaccagctgctgcagatcggagtcaagtcagagttccgacagaacctgggccgcatgtatctcttctatggcaacaagacctcggtgcagttccagaatttctcacccactgtggttcacccgggagacctccagactcagctggctgtgcagaccaagcgcgtggcggcgcaggtggacggcggcgcgcaggtgcagcaggtgctcaatatcgagtgcctgcgggacttcctgacgcccccgctgctgtccgtgcgcttccggtacggtggcgccccccaggccctcaccctgaagctcccagtgaccatcaacaagttcttccagcccaccgagatggcggcccaggatttcttccagcgctggaagcagctgagcctccctcaacaggaggcgcagaaaatcttcaaagccaaccaccccatggacgcagaagttactaaggccaagcttctggggtttggctctgctctcctggacaatgtggaccccaaccctgagaacttcgtgggggcggggatcatccagactaaagccctgcaggtgggctgtctgcttcggctggagcccaatgcccaggcccagatgtaccggctgaccctgcgcaccagcaaggagcccgtctcccgtcacctgtgtgagctgctggcacagcagttctgagccctggactctgccccgggggatgtggccggcactgggcagccccttggactgaggcagttttggtggatgggggacctccactggtgacagagaagacaccagggtttgggggatgcctgggactttcctccggccttttgtatttttatttttgttcatctgctgctgtttacattctggggggttagggggagtccccctccctccctttcccccccaagcacagaggggagaggggccagggaagtggatgtctcctcccctcccaccccaccctgttgtagcccctcctaccccctccccatccaggggctgtgtattattgtgagcgaataaacagagagacgctaaaaaaaaaaaa

SEQ ID NO: 28 - DNA, 1639 bp, Homo sapiens
  misc_feature: Incyte ID No 2355124CB1
cgccagtggtccaggagccgcttttttccactcgggaagacttcagagaagtctcacaaaggactcggctggctgcttttctcagtgccgaagccgcgccatgctcgttctcagaagcggcctgaccaaggcgcttgcctcacggacgctcgcgcctcaggtgtgttcatcttttgctacgggccctagacaatacgatggaacgttctatgaatttcgtacttattaccttaaaccttcaaatatgaatgcgttcatggaaaatcttaagaaaaacattcatcttcggacctcttactctgaattggttggattctggagtgtagaatttggaggcagaacgaataaagtgtttcatatttggaagtatgataattttgctcatcgaactgaagttcagaaagccttggccaaagataaggaatggcaagaacaattcctcattccaaatttggctctcattgataaacaagagagtgagattacttatctggtaccatggtgcaaattagaaaaacctccaaaagaaggagtctatgaactggccacttttcagatgaaacctggtgggccagctctgtggggtgatgcatttaaaagggcagttcatgctcatgtcaatctaggctacacaaaactagttggagtgttccacacagagtacggagcactcaacagagttcatgttctttggtggaatgagagtgcagatagtcgtgcagctgggagacataagtcccatgaggatcccagagttgtggcagctgttcgggaaagtgtcaactacctagtatctcagcagaatatgcttctgattcctacatcgttttcaccactgaaatagttttctactgaaatacaaaacatttcattaactgctataggatctgtctgctaatggtgcttaaattctcccaagaggttctcacttttatttgaaggaggtgttaagttaatttgctatgtttcttgcattatgaaggctacatctgtgctttgtaagtaccacttcaaaaaatagttctgtttactttctgcatggtatttcagtgtctgtcatacattaaaaatacttgtcactgttttaagatcttgactcttcatttgtttcagaatagctcttctactgtattctgacaactctttgctttatagcattttgttgtattcaaatgataatggtagcatttccatgcttgtgacagcatttttaagttattaatatattttatcaacctttccatcatgtctgttttcctggttttttttggttgttttttgaccagtaaaatttattttgtaataccaaataggatttaagaaaattaacgtatttctttactatggaaaaccacattgtcatttgtgacatcatctatattaaatatggttttcacattagttatttgtcacttacttggaaaatgatgctgttaggtcctggtattaaaaatctagaaaagacttgttggtttatgtgctgaaatgtctttatttataattaattttaactactatttacttcatttcggatcctgtttaacaaagatacttgagacatccatttgttttaatgaaatctgtatggatatggaaatgcttgccctaataaaagcctacatataaaaaaaaaaaaaaa

SEQ ID NO: 29 - DNA, 2074 bp, Homo sapiens
  misc_feature: Incyte ID No 2366939CB1
ccccgcctgccccaactccagacatcatgtgctccccgtttcggggatccagaagcttccagccagaggactgccaggaacgtcctgccaccgccaccggctcccatggcccacatacccagctgggggtgccccagcagcgggggcagcccccatgggcccccagtattgcgtgtgcaaggtggagctgtcagtgagtggccagaacctactggaccgggatgttacctccaagtccgaccccttctgtgtcctctttacagagaacaatggcagatggatcgagtacgacaggacagaaaccgcgatcaacaacctcaaccccgccttctccaagaagttcgtgcttgactaccacttcgaggaggtacagaagctcaagttcgcgctctttgaccaggacaagtccagtatgcggctggacgagcatgacttcctgggccagttctcctgcagcctgggcacgatcgtctccagcaagaagatcactaggcctctgctgctgctgaatgacaagcctgcggggaagggcttgattacgatcgctgcccaggagctgtccgacaaccgcgtcatcacactaagcctggcgggcaggaggctggacaagaaggacctctttgggaagtcagacccctttctggagttttataagccaggagacgatggcaagtggatgctggtccacaggactgaggtgatcaagtacacactggaccctgtgtggaagccattcacagtgcccttggtgtccctgtgtgatggggacatggagaagcccatccaggtcatgtgctacgactatgacaatgacgggggccatgacttcatcggcgagttccagacctcagtgtcacagatgtgtgaggctcgagacagcgtcccgctggagttcgagtgcatcaaccccaagaagcagaggaagaagaagaactataaaaactcgggcatcatcatcctgcgatcctgcaagataaaccgagactactccttccttgactacatcctgggaggctgccagctcatgttcaccgttggaatagactttacagcctccaacgggaatcccctcgacccttcctctttgcactatatcaaccctatgggcaccaacgaatatctgtcggccatctgggctgttgggcagatcattcaggactacgacagtgataagatgtttccagctctgggattcggggcccagttacccccagactggaaggtctcccatgagtttgccatcaacttcaaccccaccaaccccttctgctcaggtgtggatggtattgcccaggcgtactcagcttgcctgccccacatccgcttctacggtcctaccaatttctcccccatcgtcaaccacgtggcccggtttgcggcccaggccacacaacagcggacggccacgcagtacttcatcctcctcatcatcacggacggggtcatcagtgacatggaggagacacggcatgccgtggtgcaggcttccaagctgcccatgtccatcatcatcgtgggcgtgggcaatgcggacttcgctgccatggagttcctggatggggacagccgcatgctgcgctcccacacgggggaggaggcagcccgcgatattgtgcagttcgttccctttcgagagttccgcaacgcagcaaaagagaccttggccaaagctgtgctggcggagctgccccaacaagttgtgcagtatttcaagcataaaaacctgccccccaccaactcggagcccgcctgagctccagtgcccagcagcagcatgtcagctgagcctcctgccctcccccaggaacatgcacgctcactctgcttccttgtgggtggcctttttttaccgatccccttttttattttttacaaccggacctccacccccaacttcctccagcccagctgggcttcctttgttggagtcaactgttgatgcttccaggccaaactggcttcctctcctcctctccccacctttgccattcttaagtattgaatgtactttgtataattttagtggaattgttattgagaataaaatttttacaatcataaaaaaaaaaaaaaa

SEQ ID NO: 30 - DNA, 611 bp, Homo sapiens
  misc_feature: Incyte ID No 2483906CB1
atggtctaagccgggcgcctcacctgtcagccgcaccggctccagcgctcgcctctcgccctcgcttctccagcgctccttgctcgcaaggcgggggaggcggcggcccagccacgatgatacatttcatattgctcttcagtcgacaagggaaattacggctacagaaatggtacatcactctccctgataaagagaggaagaagatcacccgggaaattgttcagattattctctcccgtggtcacaggacaagcagttttgttgactggaaggagctaaaacttgtttataaaaggtatgctagtttatatttttgctgtgcaatagaaaatcaggacaatgagctcttgacgctagagattgtgcatcgttacgtggagctgctggacaaatattttggaaatgtctgtgagctggatattatctttaattttgaaaaggcttatttcatcctggacgagtttataataggtggggaaattcaggaaacatccaagaaaattgctgtcaaagccattgaagactctgatatgttacaggagacaatggaagaatacatgaacaagcctacattttaactggaaatctacttgaagactccagcacta

SEQ ID NO: 31 - DNA, 2871 bp, Homo sapiens
  misc_feature: Incyte ID No 2499488CB1
cactgggtcaaagcaccctgtattattccttgttaccattattgctccctgaagagggctctcatgtatctatgtaggataatccaacctcaacacagcggctgaaagaatctgctaaaaaataattcagtgccccaaaatacactgggctccaaataaactaaagagcaaaaattggctcatgtggcatgaaacaagtaatcgtatcccaagtaagaggacagtttccttcgtgttatcaacaggactgtaccccataagtttccaggtacatttggtaagagatggtgttttaacacgattttctgttgcaacagagtatcagaaaaatgcacagattgagaagctccggagtgagatcgtcgtattgaaggaagagctgcagctcaccaggtctgagctagaggctgcacaccatgccagtgcagtcagattctccaaggaatatgaaatgcagaaaacaaaagaggaagactttttgaagttatttgacaggtggaaagaagaagaaaaggagaaactagttgatgaaatggaaaaagtcaaggagatgtttatgaaggaatttaaagaattaacttcgaagaattcagcattagaatatcaactgtcagaaatccagaagtccaatatgcagatcaagtccaacataggcacattaaaagatgcacacgagtttaaagaagaccgttctccatatccccaggatttccataatgtcatgcagcttcttgatagtcaggaaagcaaatggacagctcgagttcaagctattcatcaagaacacaagaaagagaagggtcggctcctgtcacatatagagaaacttcgaacctcaatgatagatgatctaaatgcaagcaatgttttctataagaaaaggatagaagagctagggcagagactccaggagcagaatgagctgattataactcagagacagcagattaaagactttacctgtaatccattaaacagtatcagtgaacccaaaggaaatcctttagcctggcaggcttttgaatctcagccagctgctccagctgtgcctatgaatgccccagccctgcacactttggaaactaaatcaagtctgccaatggtgcatgaacaggcattctcgtcgcacatactggaaccaatagaagaactttcagaggaagaaaaaggaagggaaaatgaacagaaattaaataacaacaaaatgcatttaaggaaagctttgaagagtaactcctccctcactaagggactaagaacaatggtggagcagaacttgatggagaaactggaaaccttggggattaatgcagatatacgtggcatttcaagtgatcagttgcatagagtactaaaaagtgtggaatcagaaagacataagcaagaaagagaaatacctaactttcatcaaattcgagaattccttgaacatcaagtcagctgtaaaattgaggagaaagcactactctcttcagatcagtgcagtgtttctcaaatggataccctttcaactggagaagtacccaaaatgatacaacttccttccaaaaacagacaactgattagacaaaaagctgtttctactgataggacatctgttccaaaaattaagaaaaatgtcatggaagatccttttcccagaaagtcttcaactattacgacccctccttttagttcagaggaggagcaggaggacgacgacctcatccgggcatacgcatccccaggcccacttcctgtgccgccaccacaaaacaagggcagcttcgggaagaacacagtgaaaagtgacgcggacgggaccgagggaagcgaaatcgaggacactgatgattctcccaagcccgcaggagtcgccgttaaaacacctactgaaaaagttgaaaagatgtttccacatcgcaaaaatgtgaacaaaccagtcggtggaactaatgtccctgagatgtttatcaaaaaagaagaattacaagaactaaagtgtgcggatgtggaggatgaagactgggacatatcatccctagaggaagagatatctttgggaaaaaaatctgggaaagaacagaaggaacctccacctgcgaaaaatgaaccacattttgctcatgtgctaaatgcctggggcgcatttaatcctaaggggccaaagggagaaggacttcaagaaaatgaatcaagcacattaaaaagcagcttagtaactgtgactgattggagcgacacttcagatgtctaattccacatgtcagaagattattccagaagccagcagtatttcagtatcacagtgtttcagtaatttgcctccatgattctagtgcttctgccttaccgtgtttcccacagcaacacagagactgattcaaagaacaatggtctctttaatggcacccaatacagtattgaaaatcagatcatcaacagtatttcgaagcatgtaaaggtgtttaagacttccgctgctgcttaaaaataacatgtcattgaagtcataaaaagttttttcttcagaaaggtactctagtgttaagtgtatttttttcaactaattttttagtgaattttttttaaacttacagcatgttttggtttgaattactaaaactttaaaaaatatttttcttatgtatgctgtcgtatcgtaggcgtttatattataaaattctgttagtagtcttaaaattgaattggtggaaccactaatccttaaaagttagtctggttatttttcatatagaagtaagtttaatccgagtgtggtggtgttcacctttaatcccagctacttgggaggctgaggtgggaggataacttgagcacaggagttcaagaccagcgtgggcaatatagcaagactccacccctccacac

SEQ ID NO: 32 - DNA, 2123 bp, Homo sapiens
  misc_feature: Incyte ID No 2559148CB1
cggggccgagatgcggtgacctgccagcacctgccgcagccttcgtccgggagtcgccccatctctccacgcatcggggccctgtgccccttgctgctgcagccgggcaccatgtcgacctcgtccttgaggcgccagatgaagaacatcgtccacaactactcagaggcggagatcaaggttcgagaggccacgagcaatgacccctggggcccatccagctccctcatgtcagagattgccgacctcacctacaacgttgtcgccttctcggagatcatgagcatgatctggaagcggctcaatgaccatggcaagaactggcgtcacgtttacaaggccatgacgctgatggagtacctcatcaagaccggctcggagcgcgtgtcgcagcagtgcaaggagaacatgtacgccgtgcagacgctgaaggacttccagtacgtggaccgcgacggcaaggaccagggcgtgaacgtgcgtgagaaagctaagcagctggtggccctgctgcgcgacgaggaccggctgcgggaagagcgggcgcacgcgctcaagaccaaggaaaagctggcacagaccgccacggcctcatcagcagctgtgggctcaggcccccctcccgaggcggagcaggcgtggccgcagagcagcggggaggaggagctgcagctccagctggccctggccatgagcaaggaggaggccgaccagcccccgtcctgcggccccgaggacgacgcccagctccagctggcccttagtttgagccgagaagagcatgataaggaggagcggatccgtcgcggggatgacctgcggctgcagatggcaatcgaggagagcaagagggagactgggggcaaggaggagtcgtccctcatggaccttgctgacgtcttcacggccccagctcctgccccgaccacagacccctgggggggcccagcacccatggctgctgccgtccccacggctgcccccacctcggacccctggggcggcccccctgtccctccagctgctgatccctggggaggtccagcccccacgccggcctctggggacccctggaggcctgctgcccctgcaggaccctcagttgacccttggggtgggaccccagcccctgcagctggggaggggcccacgcctgatccatggggaagttccgatggtggggtcccggtcagtgggccctcagcctccgatccctggacaccggccccggccttctcagatccctggggagggtcacctgccaagcccagcaccaatggcacaacagcagccgggggattcgacacggagcccgacgagttctctgactttgaccgactccgcacggcactgccgacctccgggagcagcgcaggagagctggagctgctggcaggagaggtgccggcccgaagccctggggcgtttgacatgagtggggtcaggggatctctggctgaggctgtgggcagccccccacctgcagccacaccaactcccacgccccccacccggaagacgccggagtcattcctggggcccaatgcagccctcgtcgacctggactcgctggtgagccggccgggccccacgccgcctggagccaaggcctccaaccccttcctgccaggcggaggcccagccactggcccttccgtcaccaaccccttccagcccgcgcctcccgcgacgctcaccctgaaccagctccgtctcagtcctgtgcctcccgtccctggagcgccacccacgtacatctctccccttggcgggggccctggcctgccccccatgatgcccccgggccccccggcccccaacactaatcccttcctcctataatccagggcggaagggggcctggctccatccggctgccccattccggctccctgggagatcagtgttgtgagtgcatgtgaaatgggggatccccacccccagtgcccttccccttcctggggcccactcacactacaccctcttcctttcccaccccacctccccggagagaaactggacatggggcctggggaggggagctggccagaggaggacccctttcccgtggcattagaagggggaggggtggctggggccctcacccattccccctccctccc

SEQ ID NO: 33 - DNA, 1973 bp, Homo sapiens
  misc_feature: Incyte ID No 3321481CB1
agccgacaccgccggtcgccacgctggagtgcagtggtgcgagctcggctcactgaaacctccttctcttgggttcaagtgatttgtgtgcctcagcctctggagtagctgggatttcagccatcttcaagttggccgacagcgctgctcacctggccgcctcccctttaagacgcacacacctgggctctcacctgggcgcaggcgcttccgcagaagaaggaagcggcgccgccatcgcctcccggcgctccctccccgactcctaagtccttcggccgccaccatgtccgcctcggctgtcttcattctggacgttaagggcaagccattgatcagccgcaactacaagggcgatgtggccatgagcaagattgagcacttcatgcctttgctggtacagcgggaggaggaaggcgccctggccccgctgctgagccacggccaggtccacttcctatggatcaaacacagcaacctctacttggtggccaccacatcgaagaatgccaatgcctccctggtgtactccttcctgtataagacaatagaggtattctgcgaatacttcaaggagctggaggaggagagcatccgggacaactttgtcatcgtctacgagttgctggacgagctcatggactttggcttcccgcagaccaccgacagcaagatcctgcaggagtacatcactcagcagagcaacaagctggagacgggcaagtcacgggtgccacccactgtcaccaacgctgtgtcctggcgctccgagggtatcaagtataagaagaacgaggtcttcattgatgtcatagagtctgtcaacctgctggtcaatgccaacggcagcgtccttctgagcgaaatcgtcggtaccatcaagctcaaggtgtttctgtcaggaatgccagagctgcggctgggcctcaatgaccgcgtgctcttcgagctcactggcctttcaggcagcaagaacaaatcagtagagctggaggatgtaaaattccaccagtgcgtgcggctctctcgctttgacaacgaccgcaccatctccttcatcccgcctgatggtgactttgagctcatgtcataccgcctcagcacccaggtcaagccactgatctggattgagtctgtcattgagaagttctcccacagccgcgtggagatcatggtcaaggccaaggggcagtttaagaaacagtcagtggccaacggtgtggagatatctgtgcctgtacccagcgatgccgactcccccagattcaagaccagtgtgggcagcgccaagtatgtgccggagagaaacgtcgtgatttggagtattaagtctttcccggggggcaaggagtacttgatgcgagcccactttggcctccccagtgtggaaaaggaagaggtggagggccggccccccatcggggtcaagtttgagatcccctacttcaccgtctctgggatccaggtccgatacatgaagatcattgagaaaagtggttaccaggccctgccctgggttcgctacatcacccagagtggcgattaccaacttcgtaccagctagaagggagaagagatgggggcttgaacacggggcttccttacagccccggatgcagattttagagggagggcaggtgcgggctgtgtgtgtctgtgtgagggcaggtcctggacttggcagtttcttgctcccagcacccgccccttcctcacctcttccttattccataggctgggagagaaactctctctgcttccctcgcccttggagctttccccatccccctgattttatatgaagaaatagaagaggggcttgaagtccccctcgcgagtgccttcttgcaattacctgccttagcgggtgttgcgggtccctccttcacagccgctgagcccagaggtcccgctggcccctcctctgaattttaggatgtcattaaaaagatgaatctaaaaaaaaaaaaaaa

SEQ ID NO: 34 - DNA, 1535 bp, Homo sapiens
  misc_feature: Incyte ID No 3367918CB1
gagagacgaaaggagggggggggtggaagggagaggagggcgagcaaggggaggagaggagtgacgggggcggggggggagtagagaatggggaaggggaggagggaacgggaaggcgcagaggtgaggaagaagggtggggggaggggaaggggggaggaggcgcggaaggagagggagtgaggatgagacgagaagagcgggggtagaggggagtgaaaggaatagtgggcaagcagtgagaagaagagggaagggagccacgaatgcaaggaggcgaagggggggtgtgccagaggaaggaaaacgtggttgaagaagagaaaaccaggtggggggtgtgttgcaggacagcgaaggggggggaggagaggggggggaagaaggggatttgtataaggagggggtagacagaggatagataagagagccggccataggaccccaggcaatggtaggtttgggcgatgaggaacaggggtcaaggtggggggaagaagaattggcagaggcgtagggaataataggcccgaaggagtaatttcgggtactggggagggagagacaggagaatggcttgcgccttggaggcagaggttgcagtgtaccgagatagcgccactgcactcagtttgggggacagagtgagaacctatttattaaaaagaaaaacaggccaggggggtggctgacatatgtaatcccagcactttgggaggccgaggcaggtggatcaccagaggtcgggagttcaagatcagcctggccaacatggtgaaacctcatttttattaaaaatacaaatgcttagctgggcgtggtggcagggcgctggtaatcctgtacttgggaggctgaggcaggagaatcacttgaacccaggaggcagaggttgcagtgagcaaaaaaaaaaaaaaaaaaaacttacatcccttctccccaccatcctgttatcacaggagatctataaggacaggcagaaattctcagaacaggttttcaaagtggcctcctcagacctggtcaacatgggcatcagtgtggttagctacactctgaaggacattcacgatgaccaggactatttgcactctttggggaaggctcgaacagctcaagtccaaaaagatgcacggattggagaagcagaggccaagagagatgctgggatccgggaagctaaagccaagcaggaaaaggtgtctgctcagtacctgagtgagatcgagatggccaaggcacagagagattacgaactgaagaaggccgcctatgacatcgaggtcaacaccccgagcacaggctgacctggcctatcagcttcaggctggagtgcaatggcgtgatctcagctactgcaacctctgcctcctgggttcaagcgattctcctacttgggaggctgaggcaggagaatcacttgaacccaggaggcagaggttgcagtgagccaagatcacaccactgcactctagcctgggcgacagtgagactccacctcaaaaaaaaggaaaaaaaaaaaaaaaaaagtcccg

SEQ ID NO: 35 - DNA, 2453 bp, Homo sapiens
  misc_feature: Incyte ID No 1227327CB1
tgaagagcctagggtggggctcgcggtgtcgcggagggcgtggaagtgtgtcagggccagcgtgccggccctacggaagccgagcctgaggcgagatctcgccttttgtattttcactgactcatatttcctttactcaggctcccacatcaggacgagaaggagccctcgagttaccgtgggagctgtgggagctgccctgtgactcttaggaagatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataacagagaaaaaaaaaaaa

SEQ ID NO: 36 - DNA, 2599 bp, Homo sapiens
  misc_feature: Incyte ID No 1416292CB1
cgaagttgcaccggttgaggatggctgacattctctctcagtcagagaccctggcgtcgcaagacctcagtggggacttcaagaagccagctctgccggtgtccccagcggcgcggagtaaggccccggccagcagttcttcaaaccctgaggaggtacagaaggaagggcccactgcgttgcaggactccaattctggggagcccgacatccctcctcctcagccggactgcggtgattttaggagtctacaggaggagcagtcgcgccccacgacagcggtttcttcccctggcggtccagcccgggctcccccctaccaagagcctccatggggtggccctgccacagccccctacagcttagagaccctgaagggcggcactatccttggcacccgtagcttgaaagggacgagttactgccttttcgggaggctgtctggctgcgacgtgtgcctggagcacccttcggtgtctcggtaccacgcagtgctgcagcacagggcgtccggccctgacggagaatgcgacagcaacgggccgggcttctacctctacgatctgggaagcacccatggcacttttctcaacaaaactcgcatcccacctcgcacctactgtcgagtccacgttgggcatgttgttcgctttggaggcagcacccggctctttatcctgcagggaccagaggaagaccgagaggcagaatccgagttaacagtaacacagttgaaggaattgcgcaagcagcagcaaatattgttggagaagaagatgctaggagaagactcagatgaagaagaggaaatggatacctctgaaaggaagataaatgctggtagccaagatgatgagatgggttgcacctggggaatgggagaagatgcagtagaggatgatgctgaagagaaccctattgtcttagagtttcagcaggaaagggaggccttttatataaaggatcccaaaaaggctctccaaggcttttttgaccgagaaggagaagaattagaatatgaatttgatgaacagggacatagcacttggctctgcagggtgagattacctgtggacgattcaactggaaaacaactggtggctgaggccattcactcaggaaagaaaaaagaagcaatgatccagtgctcattggaagcttgtcggattcttgacactttgggattgcttcggcaggaagcagtatctcggaaaaggaaagccaagaactgggaagatgaagacttttatgatagtgatgatgacacatttcttgataggactggcctgattgagaagaagcgtctgaacagaatgaagaaggctggcaagattgatgagaagccagagacctttgaatcattggttgcaaaattaaatgatgctgaaagggaactttctgaaatttctgagagattgaaagcctcaagccaagttctatcagagtctccatctcaggattctttagatgcgttcatgtcagaaatgaaatcaggcagtacattagatggtgtgtcccggaagaaacttcacctgagaacttttgaactgaggaaagaacaacagagacttaaagggttaataaaaattgtaaagccagcagagattccagaactaaaaaagactgaaactcagactacaggtgcagaaaacaaagctaaaaagcttacattgcctctatttggtgccatgaaaggaggaagcaaattcaaattaaaaactggaacagtagggaagttaccccccaagcgtccagaactccctccaactctaatgagaatgaaagatgagcctgaagtagaagaggaggaggaagaggaagaggaagaagagaaagaaaaggaggagcatgaaaagaaaaaactggaggatggaagcctcagtaggccacagccagagatagagccagaagcagcagtgcaggaaatgaggcctcccacagatctcacacattttaaagaaacccaaacccatgaaaacatgtctcaacttagcgaggaagaacagaataaagattatcaagactgtagcaaaaccacttcattgtgcgcaggaccctcagcatcaaagaatgaatatgagaaaagcagaggtgaattgaagaaaaagaaaacacctggtccaggcaaacttccaccaacactttcttccaaatatcctgaagatgacccagactactgtgtgtgggtcccacctgaaggtcaaagtggagatggcagaacccatcttaatgacaagtatggctattgattgcttcagaatcccaaaagaaaaccttgtggaccatgtgacatggaatatttgggataatgtatcaaattgaatggccagagaagtttagatgattatttgtaagatctggtgactggcttttcgttctgtgttcttggcttcctaaatttatctgcccatatgattctcatgcatttgatatttatgtttaaaagtgtttatatatgtatgtaaaaagggaaccatatgttttgagaatttgtaaagtgagagacatgatcctattaaaataagaaggcaaaaatgttcctaatattttattttattttattttttttaagagacagggtcttactctgtcacctagg

SEQ ID NO: 37 - DNA, 1294 bp, Homo sapiens
  misc_feature: Incyte ID No 2611704CB1
ttcctatccacagtcacttctgttcatttttaatgaaaacctattgctgttattttctattatgagttaacggtgatgtgtgttttttaaattttgtatacttgctctcaccctaaaatataccttaagagacttttcccctgtttttctttctgctttccaagactccaggaaaaacagcttccatggcacattttgtacagggcacatctagaatgattgccgcagaaagttctacggagcataaagagtgtgctgaaccatcaacaagaaagaacttgatgaattctcttgaacaaaagataaggtgtttggaaaaacaaagaaaagagctcctggaagttaaccagcaatgggatcagcaatttagaagtatgaaagagttatatgaaagaaaggtagcagagctgaagacgaaactggacgccgcggaaagattcctcagcacgcgggagaaggatccgcatcagaggcagagaaaggacgacaggcaaagagaggacgacaggcagcgcgacctgacccgggaccggctgcagcgggaggagaaggaaaaggaacgcctaaatgaagaattacatgaattgaaagaagagaataaacttttaaagggaaaaaatactcttgcgaacaaggaaaaggaacattacgaatgtgaaataaaacgcctcaataaggctcttcaggatgccttgaatatcaagtgttcattttccgaggactgtttgaggaagtctcgagtggaattctgccatgaggagatgagaacagaaatggaagttctgaagcagcaggtgcaaatatacgaagaagacttcaaaaaggaacgatcggatcgagagagacttaatcaagagaaagaggagctacagcaaattaatgaaacttcccaatcccagttgaacaggctgaattcccagataaaagcttgtcagatggagaaagaaaaactagaaaagcaattaaaacagatgtattgcccaccctgtaactgcggcttggttttccacctgcaagatccatgggtaccaacaggccctggagctgtgcagaagcaacgggagcacccaccagactatcagtggtatgctcttgaccagcttccgccagatgtacaacacaaggcaaatggtttatcctcagtaaagaaagtccatccgtagaagtacacacacacacacatatatagtagtatatgtataatagaatatatattctaaaatgtagaatgggatgaaaactgacctgtcaaaattttgtaatcagagtcccagaaccgaaagagaaaatgatc

SEQ ID NO: 38 - DNA, 2313 bp, Homo sapiens
  misc_feature: Incyte ID No 5136672CB1
gactacgtagccggcgccttcctcttcccatcgcgcgggtcctagccaccggtgtctccttctacatccgcctctgcgccggctgccacccgcgctccctccgccgccgccgccttgctgctgctcaaagctgctgccgccccttgggctaaaaggttttcaaatggaacattttgatgcatcacttagtacctatttcaaggcattgctaggccctcgagatactagagtaaaaggatggtttcttctggacaattatatacccacatttatctgctctgtcatatatttactaattgtatggctgggaccaaaatacatgaggaataaacagccattctcttgccgggggattttagtggtgtataaccttggactcacactgctgtctctgtatatgttctgtgagttagtaacaggagtatgggaaggcaaatacaacttcttctgtcagggcacacgcaccgcaggagaatcagatatgaagattatccgtgtcctctggtggtactacttctccaaactcatagaatttatggacactttcttcttcatcctgcgcaagaacaaccaccagatcacggtcctgcacgtctaccaccatgcctcgatgctgaacatctggtggtttgtgatgaactgggtcccctgcggccactcttattttggtgccacacttaatagcttcatccacgtcctcatgtactcttactatggtttgtcgtcagtcccttccatgcgtccatacctctggtggaagaagtacatcactcaggggcagctgcttcagtttgtgctgacaatcatccagaccagctgcggggtcatctggccgtgcacattccctcttggttggttgtatttccagattggatacatgatttccctgattgctctcttcacaaacttctacattcagacctacaacaagaaaggggcctcccgaaggaaagaccacctgaaggaccaccagaatgggtccatggctgctgtgaatggacacaccaacagcttttcacccctggaaaacaatgtgaagccaaggaagctgcggaaggattgaagtcaaagaattgaaaccctccaaaccacgtcatctgattgtaagcacaatatgagttgtgccccaatgctcgttaacagctgctgtaactagtctggcctacaatagtgtgattcatgtaggacttctttcatcaattcaaaacccctagaaaacgtatacagattatataagtagggataagatttctaacatttctgggctctctgacccctgcgctagactgtggaaagggagtattattatagtatacaacactgctgttgccttattagttataacatgataggtgctgaattgtgattcacaatttaaaaacactgtaatccaaacttttttttttaactgtagatcatgcatgtgattgtaaatgtaaatttgtacaatgttgttatggtagagaaacacacatgccttaaaatttaaaaagcagggcccaaagcttattagtttaaattagggtatgtttcaagtttgtattaatttgtaatagctctgtttagaaaaaatcaaagaccatgatttatgaaactaatgtgacataatttccagtgacttgttgatgtgaaatcagacacggcaccttcagttttgtactattggctttgaatcaagcaggctcaaatctagtggaacagtcagtttaactttttaacagatcttatttttttattttgagtgccactattaatgtaaaaaggggggggctctacagcagtcgtgatgaaacttaaatatatattctttgtcctcgagattttaggaagggtgtagggtgagtaggccatttttaatttctgaagtgctaagtgtttttatacagcaaacaaaaagtcaattttgctttccaccagtgcgagagaggatgtatacttttcaagagagatgattgcctatttaccgtttgacagagtcccgtagatgagcaatggggaactggttgccagggtctaaatttggattgatttatgcactgttatctgttttgacacagatttccttgtaaaatgtgcctagtttaccaaaattaacaaagggggggaaaggaccttagaactttttaaggtaaaatcaaatatagctacagcataagagaatcgagaaatttgatagaggtaacttgtttaatgtaaatctaatagtacttgtaatttctttctgcttagaatctaaagatgtgtttagaacctccttgttaaaataatagactgctattcataaatcacatctcacacatttggggcagtggt